(19) United States
(12) Patent Application Publication
(10) Pub. No.: US 2023/0184746 A1
(43) Pub. Date: Jun. 15, 2023

(54) HIGH-SENSITIVITY DETECTION OF GPCR ACTIVITY AND USES THEREOF

(71) Applicant: Firmenich Incorporated, Plainsboro, NJ (US)

(72) Inventors: Guy Servant, San Diego, CA (US); Mark Williams, San Diego, CA (US); Nicole Servant, San Diego, CA (US)

(21) Appl. No.: 17/793,615

(22) PCT Filed: Jan. 21, 2021
(86) PCT No.: PCT/US2021/014322

§ 371 Date: Jul. 18, 2022

Related U.S. Application Data
(60) Provisional application No. 62/964,283, filed on Jan. 22, 2020.

(51) Int. Cl. G01N 33/50

(52) U.S. Cl. CPC G01N 33/5041; G01N 2333/726

(57) ABSTRACT

The present disclosure relates generally to methods and materials for the identification of compounds that modulate (e.g., enhance, agonize, or antagonize) G protein-coupled receptor (GPCRs), such as those involved in taste. Examples of such GPCR include the TAS1R proteins and the TAS2R proteins that are involved in sensing sweet taste. In certain aspects, the disclosure provides cell-based and label-free assays that use highly sensitive biosensors to detect candidate taste modulator compounds by their alteration of GPCR activity. In some embodiments, such cell-based and label-free assays may be used to detect changes in cell mass distribution, cell adhesion, or cell morphology, resulting from contact with a candidate taste modulator compound. In some embodiments, such assays detect GPCR activation with greatly improved sensitivity compared to conventional assay methods.

### REFERENCE TO RELATED APPLICATIONS

This application claims the benefit of U.S. Provisional Application No. 62/964,283, filed Jan. 22, 2020 and U.S. Provisional Application No. 62/966,697, filed Jan. 28, 2020, both incorporated herein by reference in their entirety.

### TECHNICAL FIELD

The present disclosure relates generally to methods and materials for the identification of compounds that modulate (e.g., enhance, agonize, or antagonize) G protein-coupled receptor (GPCRs), such as those involved in taste. Examples of such GPCR include the TAS1R proteins and the TAS2R proteins that ae involved in sensing sweet, umami and bitter taste. In certain aspects, the present disclosure also provides cell-based and label-free assays that use highly sensitive biosensors to detect candidate taste modulator compounds by their alteration of GPCR activity. In some embodiments, such cell-based and label-free assays may be used to detect changes in cell mass distribution, cell adhesion, or cell morphology, resulting from contact with a candidate taste modulator compound. In some embodiments, such assays detect GPCR activation with greatly improved predictivity compared to conventional assay methods. For example, in some instances, it is possible to detect GPCR activation coupled through endogenous G proteins rather than having use overexpressed promiscuous G proteins, also known as forced coupling,. In some cases, use of cells expressing only the receptor of interest but not overexpressing a promiscuous G proteins more closely mimics taste signaling in vivo, thereby increasing the likelihood that candidate modulator compounds identified using these assays will have taste modulatory activity in vivo.

### DESCRIPTION OR RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates.

Taste transduction occurs when certain substances in foods, beverages, and other ingestible materials modulate taste receptors, which are typically expressed in cells that make up taste buds. Taste cells are located principally on the tongue, but are also located in other portions of the mouth, and taste-like cells are also located in the upper digestive system. Modulation of the taste receptors induces the nervous system to communicate sensory information to effector systems that are involved in the regulation of appetite, immune responses, gastrointestinal motility, among other things.

Advances in biotechnology have permitted the identification and cloning of many of the taste receptors involved in taste transduction. In general, two types of specific taste receptors located at the surface of taste receptor cells (TRCs) on the tongue are generally responsible for the transduction of taste associated with bitterness, sweetness, and umami These types of specific taste receptors are often referred to as TAS1R and T2R. Receptors of the TAS1R type are generally associated with sweet and umami taste, while receptors of the TAS2R type are generally associated with bitter taste. Once these specific taste receptors are identified and cloned, it may be possible to express them in a recombinant cell together with a G protein, such as the promiscuous G proteins Gα15 or Gα16. By expressing the receptors into recombinant cells, one may use these cells to create an in vitro system that simulates the in vivo functioning of TRCs. In some cases, it may be desirable to co-express multiple different taste receptors on a single recombinant cell, resulting in a hetero-oligomeric taste receptor system.

Recombinant cells expressing one or more taste receptors can be used for in vitro screening assays for identification of compounds that activate taste receptor signaling and therefore are likely to modulate taste. For example, recombinant cells expressing a taste receptor can be contacted with various candidate modulatory compounds, and changes in receptor activation (detectable through a variety of different methods) indicate compounds that are potentially useful as flavor-affecting additives, for example in foods, beverages, medicines, and the like.

Thus, it is possible to build assays that use recombinant cells transformed with genes encoding a functional taste receptor and a promiscuous G protein to screen for various compounds that may function as taste modulators. The transformed cells in such assays respond sensitively and specifically to particular taste-affecting compounds, and the resulting response is detected by fluorescent dyes that respond to changes in intracellular calcium. Even so, such assay methods face certain limitations. In particular, such assays may tend to give false positives, or may fail to provide a sufficiently strong response to compounds that otherwise induce a strong response in vivo. Thus, these kinds of assays must still be used in combination with secondary assays and human taste tests. Such secondary tests consume precious time and resources. And the risk of overlooking an otherwise-active compound remains. So, while the advent of such assays has advanced the discovery and identification of compounds that modulate sweet, bitter, and umami tastes, their use is limited to that of preliminary screening. Thus, there is a continuing need to discover ways to improve such cell-based assays so that the output of these assays is more consistent with the actual functioning of TRCs in vivo, thereby eliminating the need for secondary screening and reducing the risk that active and valuable compounds are overlooked.

### SUMMARY

The present disclosure relates to the development cell based assays for or more taste receptors, which do not include an overexpressed G protein and a label to enable detection. Instead, the transformed recombinant cells only expresses the receptor(s) of interest and are plated on speciliazied biosensor plates that can be read using highly sensitive plate readers measuring changes in mass redistribution or impedance. In general, when potential taste-affecting compounds are screened using assays comprising such cells, the assays come much closer than traditional G protein overexpressing and label-based assays to determining how such potential taste-affecting compounds would actually affect the TRCs in vivo. Thus, in many instances, such assays provide false positives at a much lower rate as traditional G protein overexpressing and label-based assays and tend not to overlook otherwise-active compounds to the same degree as traditional G protein overexpressing and label-based assays.

In a first aspect, the disclosure provides a cell-based method of detecting a taste receptor modulating compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with a test compound that potentially modulates the activity of the taste receptor; (c) measuring a functional effect of the test compound on the eukaryotic cell using an assay method that comprises using a biosensor to detect or measure a change in cell mass distribution, impedance, or a combination thereof; and (d) identifying the test compound as a taste receptor modulator based on the functional effect. In some embodiments, the eukaryotic cell expresses a G protein that functionally couples to the taste receptor.

In a second aspect, the disclosure provides a cell-based method of detecting a taste receptor modulating compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with a test compound that potentially modulates the activity of the taste receptor; (c) measuring a functional effect of the test compound on the eukaryotic cell using an assay method that comprises detecting or measuring a change in GPCR activity, wherein the change in GPCR activity is not indicated through the use of a label or a dye; and (d) identifying the test compound as a taste receptor modulator based on the functional effect. In some embodiments, the eukaryotic cell expresses a G protein that functionally couples to the taste receptor.

In a third aspect, the disclosure provides a cell-based method of detecting a taste receptor modulator compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with an identified taste receptor modulator and a test compound that potentially modulates an effect of the identified taste receptor modulator on activity of the taste receptor; (c) measuring a functional effect of the test compound on the eukaryotic cell using an assay method that comprises using a biosensor to detect or measure a change in cell mass distribution, impedance, or a combination thereof; and (d) identifying the test compound as a taste receptor modulator based on the functional effect. In some embodiments, the eukaryotic cell expresses a G protein that functionally couples to the taste receptor.

In a fourth aspect, the disclosure provides a cell-based method of detecting a taste receptor modulator compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with an identified taste receptor modulator and a test compound that potentially modulates an effect of the identified taste receptor modulator on activity of the taste receptor; (c) measuring a functional effect of the test compound on the eukaryotic cell using an assay method comprising detecting or measuring a change in GPCR activity, wherein the change in GPCR activity is not indicated through the use of a label or a dye; and (d) identifying the test compound as a taste receptor modulator based on the functional effect. In some embodiments, the eukaryotic cell expresses a G protein that functionally couples to the taste receptor.

In some embodiments of any of the foregoing aspects, the assays disclosed herein detect changes in one or more of cell mass distribution, cell adhesion, and cell morphology resulting from contact with a test compound, which may be detected using optical waveguide grating or impedance-based grating. Non-limiting examples of commercially available biosensors that may be used include SRU BIND, CELL KEY, ECIS, EPIC, RT-CES, and XCELLIGENCE.

In some embodiments of the foregoing aspects, the eukaryotic cell stably or transiently expresses TAS1R or TAS2R receptors.

In some embodiments of the foregoing aspects, the eukaryotic cell expresses a TAS1R receptor comprising TAS1R1, TAS1R2, TAS1R3, or mixtures thereof, or TAS1R1 and TAS1R3, or TAS1R2 and TAS1R3. In some embodiments of the foregoing aspects, the eukaryotic cell expresses a

TAS2R receptor comprising hT2R1, hT2R3, hT2R4, hT2R5, hT2R7, hT2R8, hT2R9, hT2R10, hT2R13, hT2R14, hT2R16, hT2R44, hT2R51, hT2R54, hT2R55, hT2R61, hT2R64, hT2R65, hT2R67, hT2R71, hT2R75, hT2R76, or mixtures thereof.

In some embodiments of the foregoing aspects, the eukaryotic cell comprises an insect cell, amphibian cell, yeast cell, worm cell, or mammalian cell.

In some embodiments of the foregoing aspects, the eukaryotic cell comprises a CHO cell, a mouse macrophage, U2OS cells, HeLa cell, or a BHK cell.

In some embodiments of the foregoing aspects, the eukaryotic cell is contained in a multiwell assay plate or adhered to a substrate.

In some embodiments of the foregoing aspects, the assay or assay kit is amenable to high-throughput screening.

In some embodiments of the foregoing aspects, the outcome of the assay is indicative of whether a compound shown to have an effect on TAS1R or TAS2R receptor activity in a eukaryotic cell has a taste modulator effect in a human or non-human animal subject.

In some embodiments of the foregoing aspects, the first compound used for identifying a second compound that modulates the effect of the first compound on TAS1R or TAS2R receptor activity is monosodium glutamate, L-aspartate, L-glutamate, a natural or artificial sweetener, saccharin, sucralose, rebaudioside A, glucose, sucrose, sorbitol, xylose, dextran, aspartame, monellin, cyclamate, fructose, treholose, D-tryptophan, xorbitol, xylitol, L-tryptophan, quinine, denatonium, lidocaine, caffeine, andrographolide, dilthiazem, chloroquine, ofloxacin, oxyphenonium, aristolocihc acid, ranitidine, cycloheximide, strychnine, salicin, phenylthiocarbamide, or mixtures thereof.

In certain other related aspects, the disclosure provides assays for identifying modulators of TAS1R or TAS2R taste receptors wherein at least one TAS1R to TAS2R is stably or transiently expressed in a cell, such as a mammalian cell line such as U2OS cells, and the modulator is identified based on its effect on G protein mediated signaling pathways that affect cell mass distribution, cell adhesion, or cell morphology as measured using optical waveguide grating or impedance-based grating.

In a fifth aspect, the disclosure provides compounds, wherein the compounds are the taste receptor modulators identified by the methods of any of the foregoing aspects or embodiments thereof.

In a sixth aspect, the disclosure provides uses of the taste receptor modulator identified by the methods of any of the foregoing aspects or embodiments thereof to modify a taste of a comestible composition. In some embodiments thereof, the comestible composition is not a naturally occurring composition. In some embodiments thereof, modifying the taste of a comestible composition comprises enhancing a sweet taste of the comestible composition, enhancing an umami taste of a comestible composition, or inhibiting a bitter taste of a comestible composition. In some embodiments, the comestible composition is a food product, a beverage product, a pharmaceutical composition, a cosmetic composition, an oral care composition, or a dentrifice.

In a seventh aspect, the disclosure provides comestible compositions comprising the taste receptor modulator identified by the methods of any of the foregoing aspects or embodiments thereof. In some embodiments thereof, the comestible composition is a naturally occurring composition. In some embodiments thereof, the comestible composition is not a naturally occurring composition. In some embodiments, the comestible composition is a food product, a beverage product, a pharmaceutical composition, a cosmetic composition, an oral care composition, or a dentrifice.

Further aspects and embodiments are set forth in the Detailed Description, the figures, the abstract, and the claims.

### DETAILED DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

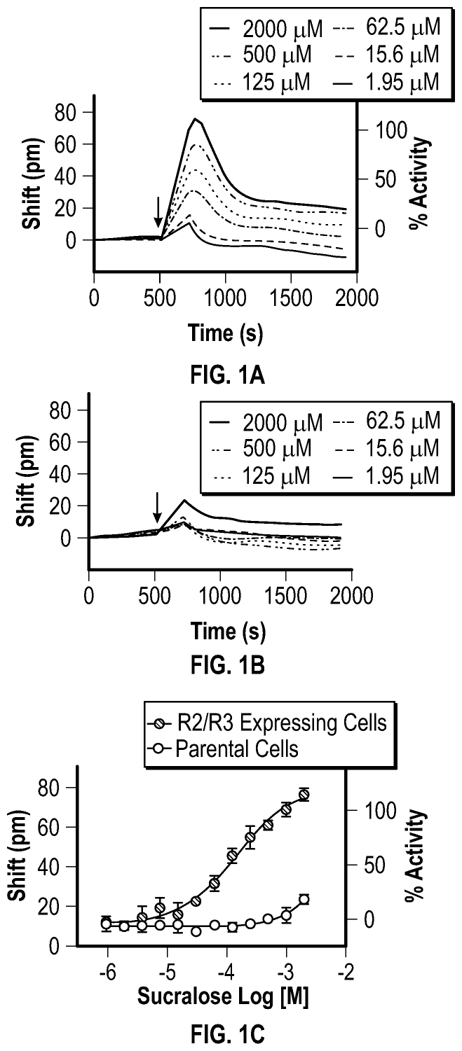

FIGS. 1A, 1B and 1C show a sweetener-induced DMR response in U2OS cells expressing the sweet taste receptor but no response with the parental cells.

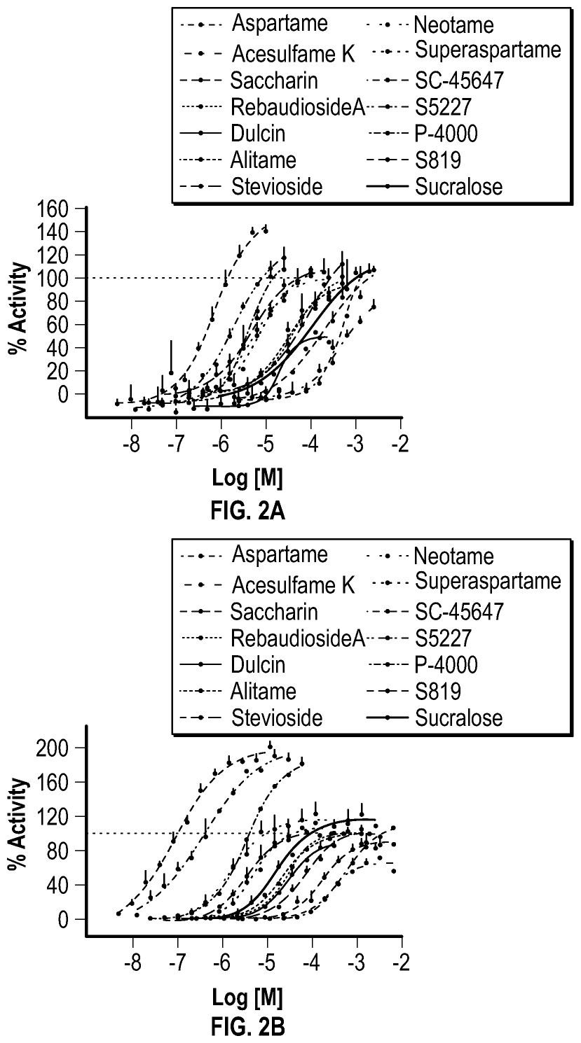

FIGS. 2A and 2B show several sweeteners-induced DMR responses in U2OS cells expressing the sweet taste receptor vs the same sweetener response detected on FLIPR in cells also overexpressing a promiscuous G protein.

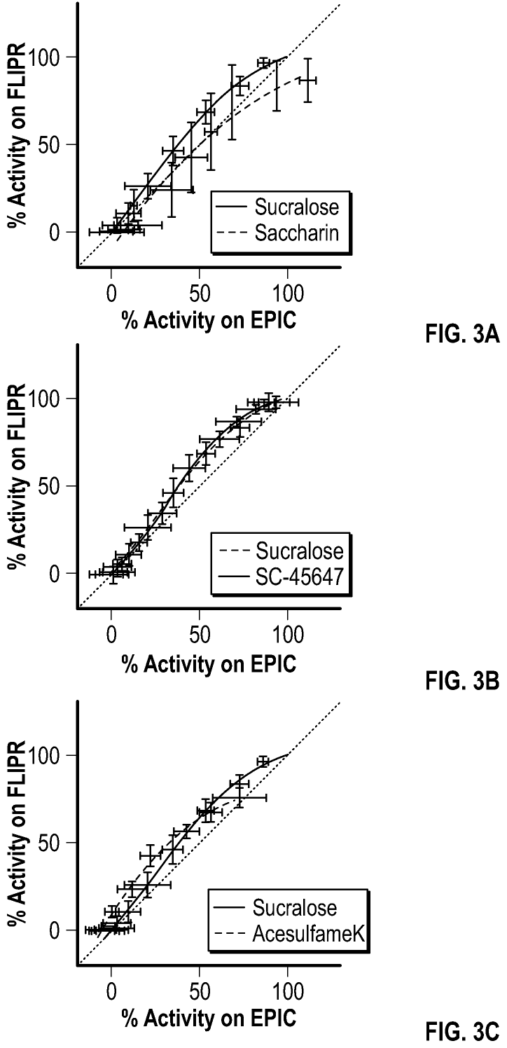

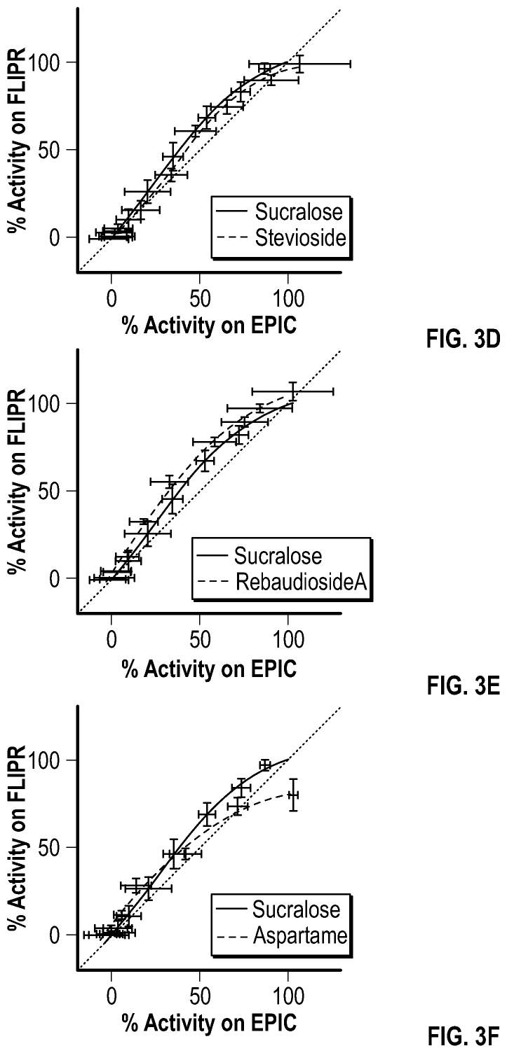

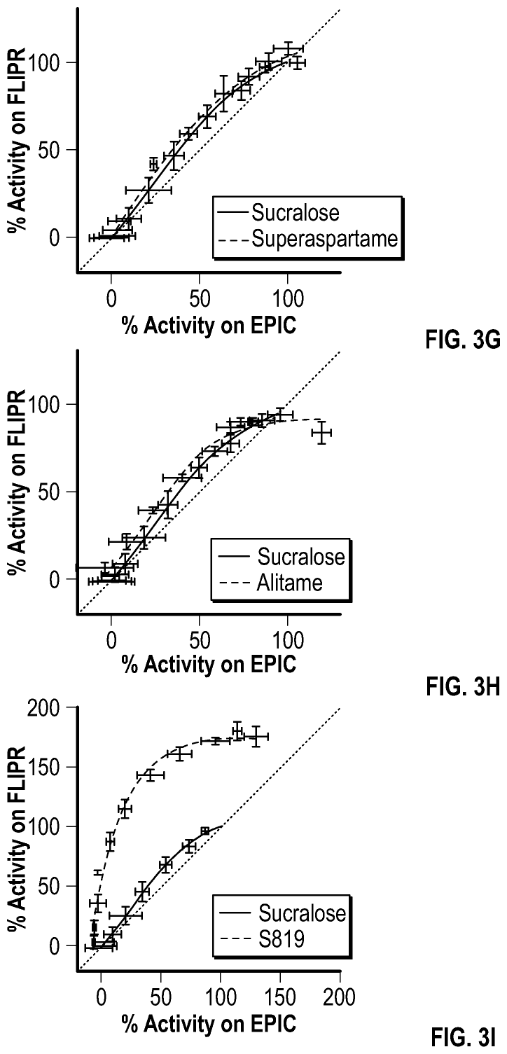

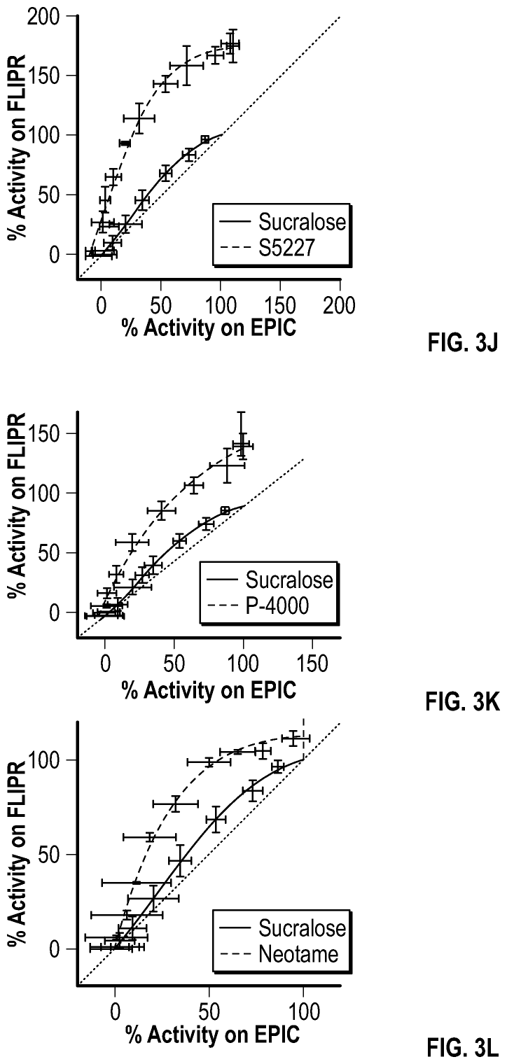

FIGS. 3A, 3B, 3C, 3D, 3E, 3F, 3G, 3H, 3I, 3J, 3K, and 3L show bias-plot analysis of the effects of sweeteners in the EPIC (DMR) assay vs the promiscuous G protein FLIPR assay. S819, S2227, P-4000 and Neotame show a clear bias for the FLIPR assay.

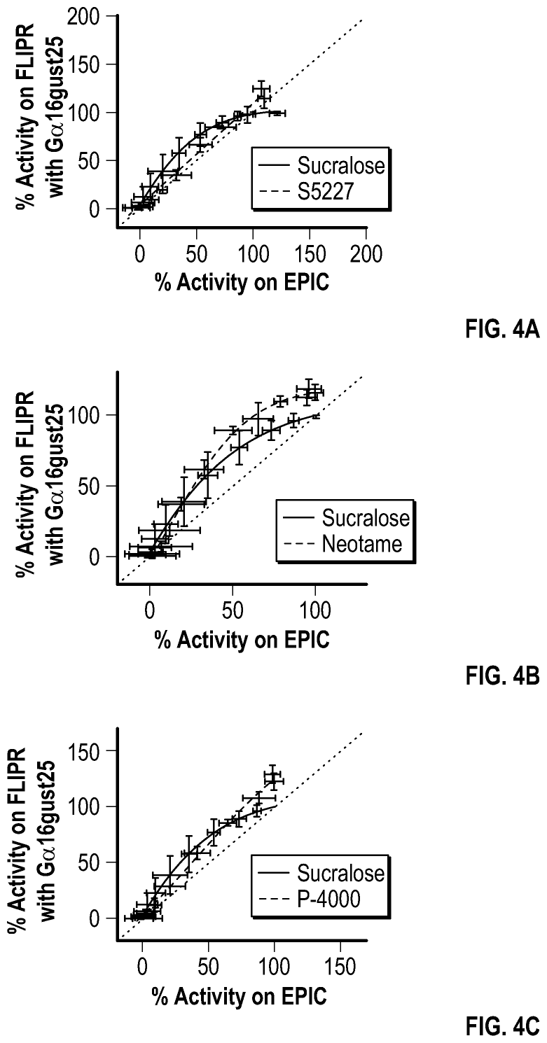

FIGS. 4A, 4B, and 4C hows that addition of a c-terminal tail transducing to the promiscuous G protein G16 abbrogates bias.

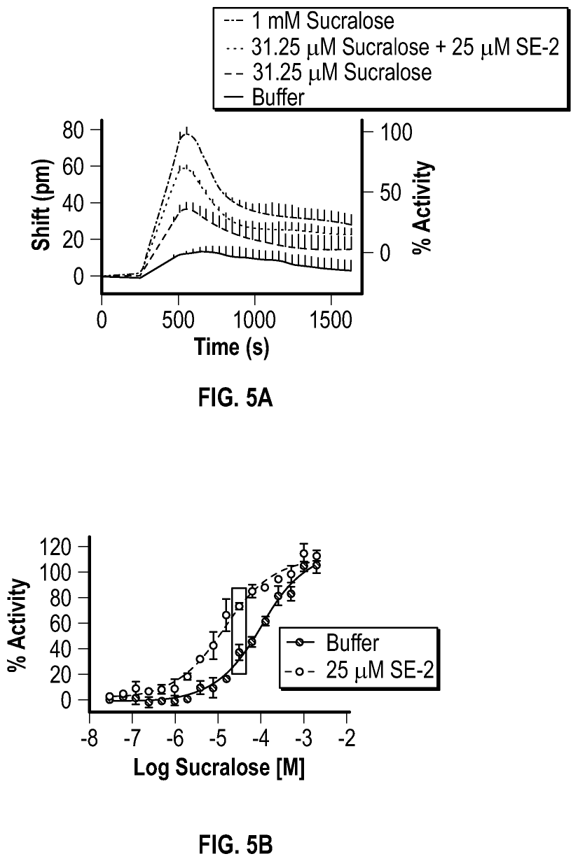

FIGS. 5A and 5B show that the effect of a sweet taste receptor positive allosteric modulator can be detected in the DMR assay in cell expressing the sweet taste receptor without an overexpressed G protein .

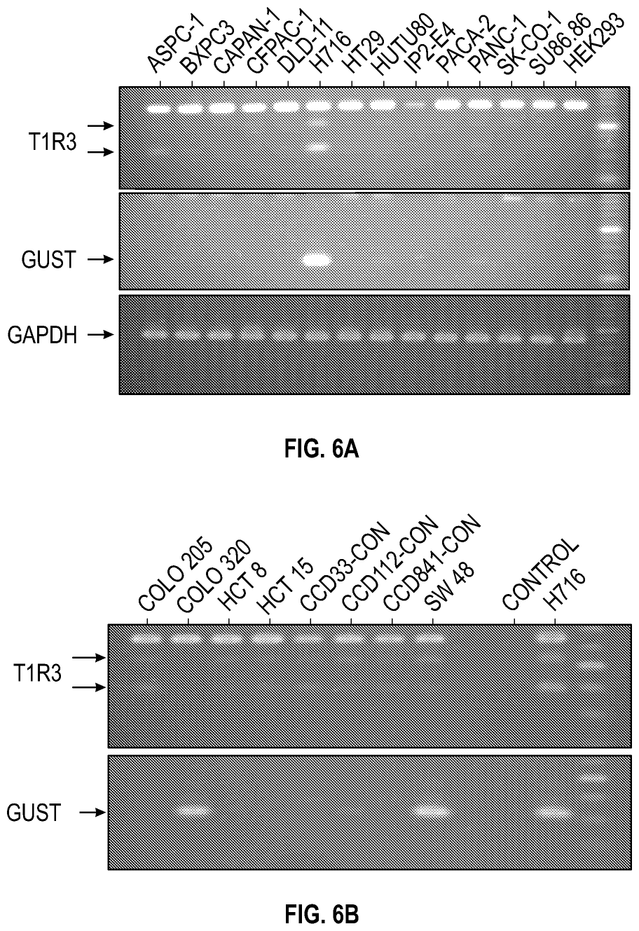

FIGS. 6A and 6B show RT-PCR results for expression of TAS1R3 and gustducin in different cell lines.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions and General Information

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “functional fragment” or “fragment” refers to a portion of a protein or polypeptide sequence to which the test compound binds. Polypeptide sequences often contain certain amino acids that do not actively participate in binding, but which may serve other purposes. In some instances, these non-functioning parts of the polypeptide sequence can be removed or partially replaced, while leaving the functional portion of the sequence intact. These modified proteins are said to comprise a fragment or functional fragment of the original polypeptide sequence.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Some abbreviations used in this application are as follows: cAMP: 3′5′-cyclic adenosine monophsphate, TRCs: Taste receptor cells, GPCRs: G protein-coupled receptors, MSG: Monosodium glutamate, PDE: phosphodiesterase; MAPK: Mitogen activated protein kinase, IMP: inosine monophosphate, PTX: pertussis toxin, EGF: Epidermal growth factor, PKC: Protein kinase C, RTKs: Receptor tyrosine kinases, PKA: Protein kinase A, ACs:

Adenylyl cyclases, cNMP: cyclic nucleotide monophosphate, CREB: cAMP response element-binding protein, PLCβ2: Phospholipase Cβ2, Trp: Transient receptor potential, TCR: taste cell receptor.

The term “taste cells” include neuroepithelial cells that are organized into groups to form taste buds of the tongue, e.g., foliate, fungiform, and circumvallate cells (see, e.g., Roper et al., Ann. Rev. Neurosci. 12:329-353 (1989)) (31). Taste cells are also found in the palate and other tissues, such as the esophagus and the stomach.

The term “TAS1R” refers to one or more members of a family of G protein-coupled receptors that are expressed in taste cells such as foliate, fungiform, and circumvallate cells, as well as cells of the palate, and esophagus (see, e.g., Hoon et al., Cell, 96:541-551 (1999), herein incorporated by reference in its entirety). The definition of “TAS1R” should further be construed based on DNA and amino acid sequences disclosed in the Senomyx and University of California patent applications and publications incorporated by reference herein. Members of this family are also referred to as GPCR-B3 and TR1 in WO 00/06592 as well as GPCR-B4 and TR2 in WO 00/06593. GPCR-B3 is also herein referred to as rT1R1, and GPCR-B4 is referred to as rT1T2. Taste receptor cells can also be identified on the basis of morphology, or by the expression of proteins specifically expressed in taste cells. TAS1R family members may have the ability to act as receptors for sweet or umami taste transduction, or to distinguish between various other taste modalities. The present assignee and its exclusive licensor, the University of California, have filed a number of patent applications relating to human and rodent TAS1R taste receptors. Specifically, Senomyx has filed patent applications Ser. No. 09/897,427, filed on Jul. 3, 2001 (now U.S. Pat. No. 6,955,887), U.S. Ser. No. 10/035,045 filed on Jan. 3, 2002 (now U.S. Pat. No. 7,241,880), U.S. Ser. No. 10/179,373, filed on Jun. 26, 2002 (now U.S. Pat. No. 7,368,285), and U.S. Ser. No. 09/799,629, filed on Mar. 7, 2001 (now U.S. Pat. No. 7,244,835). Additionally, the University of California has filed a number of applications exclusively licensed by Senomyx including U.S. Ser. No. 09/361,631, filed Jul. 27, 1999 (now U.S. Pat. No. 6,383,778) and U.S. Ser. No. 09/361,652, filed on Jul. 27, 1999 (published as US 2003/0036630) which relates to cloned rat, mouse and human TAS1R1 and TAS1R2 genes and the use of the genes and corresponding polypeptides to identify TAS1R modulators. Each of the foregoing patents and patent applications are incorporated by reference in their entirety herein (including the sequences contained therein).

“TAS1R” nucleic acids encode a family of GPCRs with seven transmembrane regions that have “G protein-coupled receptor activity,” e.g., they may bind to G proteins in response to extracellular stimuli and promote production of second messengers such as IP3, cAMP, cGMP, K+, and Ca2+ via stimulation of enzymes such as phospholipase C and adenylate cyclase (for a description of the structure and function of GPCRs, see, e.g., Fong, TM Cells Signal. 8(3):217-224 (1996) (33) and Baldwin, et al., J. Mol. Biol. 272(1):144-164 (1997) (34). A single taste cell may contain many distinct TAS1R polypeptides.

The term “TAS1R” family therefore refers to polymorphic variants, alleles, mutants, and interspecies homologus that: (1) have at least 35 to 50% amino acid sequence identity, optionally 60, 75, 80, 85, 90, 95, 96, 97, 98, or 99% amino acid sequence identity to a TAS1R polypeptide, over a window of 25 amino acids, optionally 50-100 amino acids; (2) specifically bind to antibodies raised against an immunogen comprising an amino acid sequence selected from the group consisting of the TAS1R polypeptide sequence disclosed in the patent applications incorporated by reference herein and conservatively modified variants thereof; (3) are encoded by a nucleic acid molecule which specifically hybridize (with a size of at least 100, optionally at least 500-1000 nucleotides) under stringent hybridization conditions to a sequence selected from the group consisting of the TAS1R nucleic acid sequences contained in the applications incorporated by reference in their entirety herein, and conservatively modified variants thereof; or (4) comprise a sequence at least 35 to 50% identical to an amino acid sequence selected from the group consisting of the TAS1R amino acid sequence identified in the patent applications incorporated by reference in their entirety herein.

The term “TAS2R” refers to one or more members of a family of G protein coupled receptors that are expressed in taste cells, specifically, the tongue and palate epithelia. In particular, TAS2R includes the particular genes identified in the Senomyx and University of California applications relating to TAS2Rs incorporated by reference in their entirety herein. TAS2Rs are genetically linked to loci associated with bitter taste perception in mice and humans. More specifically, the term “TAS2R” and terms including TAS2R, e.g., TAS2R04 or TAS2R05 refers generally to isolated TAS2R nucleic acids, isolated polypeptides encoded by TAS2R nucleic acids, and activities thereof. TAS2R nucleic acids and polypeptides can be derived from any organism. The terms “TAS2R” and terms including “TAS2R” also refer to polypeptides comprising receptors that are activated by bitter compounds, and to nucleic acids encoding the same. Thus both TAS1Rs and TAS2Rs comprise different families of chemosensory GPCRs. Sequences of various TAS2Rs are also contained in the Appendix that precedes the claims. The present assignee has filed a number of patent applications relating to various TAS2R genes and the corresponding polypeptides and their use in assays, such as a high throughput cell-based assays for identifying compounds that modulate the activity of TAS2Rs. These Senomyx applications, i.e., U.S. Ser. No. 09/825,882, filed on Apr. 5, 2001, U.S. Ser. No. 10/191,058 filed Jul. 10, 2002 and U.S. Provisional Application Ser. No. 60/398,727, filed on Jul. 29, 2002 all incorporated by reference in their entireties herein. Additionally, the present assignee has exclusively licensed patent applications relating to TAS2R genes which were filed by the University of California i.e., U.S. Ser. No. 09/393,634, filed on Sep. 10, 1999 (recently allowed) and U.S. Ser. No. 09/510,332, filed Feb. 22, 2000, that describe various mouse, rat and human TAS2R sequences and the use thereof in assays for identifying molecules that modulate specific TAS2Rs and which modulate (enhance or block) bitter taste. These applications and the sequences contained therein are also incorporated by reference in their entireties herein.

G proteins are heterotrimeric proteins composed of a single α subunit complexed with the βγ dimer. Molecular cloning has resulted in the identification of at least 18 distinct a subunits, 5 β subunits, and 12 β subunits. G proteins are usually divided into four subfamilies Gi, Gs , Gq, and G12 based on the sequence similarity of the Gα subunit. G proteins are categorized based upon their effect on cyclic AMP (“cAMP”) in cells. For example, Gs (stimulatory) stimulates the enzyme adenylyl cyclase. By contrast, Gi (inhibitory) (and Gz and G0) inhibit this enzyme. Adenylyl cyclase catalyzes the conversion of ATP to cAMP. Thus, constitutively activated GPCRs that couple Gi (or Gz and G0) protein associated with a decrease in cellular levels of cAMP. See, generally, “Indirect Mechanisms of Synoptic Transmission,” Chapter 8, From Neuron to Brain (3rd Edition), Nichols, J. G. et al eds., Sinauer Associates, Inc. (1992). Several lines of evidence suggest that the interaction between a given GPCR and its cognate G protein involves multiple sites of contact on both proteins. All three intracellular loops as well as the carboxyl terminal tail of the receptor have been implicated. The GPCR is though to interact with all three subunits of the G protein. As the receptor-G protein interaction can be disrupted by a number of treatments that block the carboxyl terminus, including pertussis toxin-catalyzed ADP-ribosylation of Ga and binding of monoclonal antibodies, the carboxy terminal region of the Ga subunit has been the most intensely investigated contact site. These studies have shown that the Ga carboxy-terminal region is important not only to the interaction, but also plays a critical role in defining receptor specificity (Hamm et al., Science 241: 832-5 (1988); Osawa et al., J. Biol. Chem. 270: 31052-8 (1995); Garcia et al., EMBO 14: 4460-9 (1995); Sullivan et al., Nature 330: 758-760 (1987); Rasenick et al., J. Biol. Chem. 269: 21519-21525 (1994); West et al., J. Biol. Chem. 260: 14428-30 (1985); Conklin et al., 1993, Nature 363: 274-276; Conklin et al., Mol. Pharmacol. 50: 885-890 (1996)). Furthermore, it has been shown that peptides corresponding to the carboxy terminal region of a Gαi subunit can block GPCR signaling events (Hamm et al., Science 241: 832-5 (1988); Gilchrist et al., J. Biol. Chem 273: 14912-19 (1998)). The present assignee has also obtained U.S. Pat. No. 7,022,488, filed on Feb. 3, 2004, that disclosed that TAS1R and TAS2R receptors functionally couple with Gi proteins, such as Gαi, when expressed in human embryonic kidney cells. TAS1R and TAS2R receptor activity was measured by assaying MAPK activity, cAMP accumulation, or adenylyl cyclase activity. The GTP-bound form of Gαi can directly interact and inhibit up to 6 different types of adenylyl cyclase. ERK1/2 is activated by Gq, Gs, and Gi-coupled GPCRs (Liebmann et al (1996); Pierce et al., Oncogene 20(13): 1532-1539 (2001); Gutkind, J. S., J. Biol Chem 273(4): 1839-42 (1998)) and, depending on the cellular context, several signaling pathways can be triggered to activate ERK1/2. Specifically, it is thought that Gi-coupled GPCRs activate ERK1/2 mainly via the free (activated) Gβγ subunits (Crespo et al. Nature 369: 418-20 (1994); Faure et al., J. Biol Chem. 269(11): 7852-7854 (1999)) that recruit and activate soluble tyrosine kinases of the Src (Gutkind, 1998) and Bruton families (Wan et al., J. Biol Chem. 272(27): 17209-15 (1997)) or somehow transactivate receptor tyrosine kinases (RTKs) at the cell surface to initiate the cascade Liebmann et al. (2001); Wu et al. Bioch. Biophys Acta. 1582:100-106 (2002)).

Topologically, certain chemosensory GPCRs have an “N-terminal domain;” “extracellular domains;” “transmembrane domains” comprising seven transmembrane regions, and corresponding cytoplasmic, and extracellular loops; “cytoplasmic domains,” and a “C-terminal domain” (see, e.g., Hoon et al., Cell, 96:541-551 (1999); Buck & Axel, Cell, 65:175-187 (1991)). These domains can be structurally identified using methods known to those of skill in the art, such as sequence analysis programs that identify hydrophobic and hydrophilic domains (see, e.g., Stryer, Biochemistry, (3rd ed. 1988); see also any of a number of Internet based sequence analysis programs. Such domains are useful for making chimeric proteins and for in vitro assays in embodiments of the present disclosure, e.g., ligand binding assays.

The term “extracellular domains” therefore refers to the domains of TAS1R and TAS2R polypeptides that protrude from the cellular membrane and are exposed to the extracellular face of the cell. Such domains generally include the “N terminal domain” that is exposed to the extracellular face of the cell, and optionally can include portions of the extracellular loops of the transmembrane domain that are exposed to the extracellular face of the cell, i.e., the loops between transmembrane regions 2 and 3, between transmembrane regions 4 and 5, and between transmembrane regions 6 and 7.

The “N-terminal domain” region starts at the N-terminus and extends to a region close to the start of the first transmembrane domain. More particularly, in one embodiment, this domain starts at the N-terminus and ends approximately at the conserved glutamic acid at amino acid position 563 plus or minus approximately 20 amino acids. These extracellular domains are useful for in vitro ligand-binding assays, both soluble and solid phase. In addition, transmembrane regions, described below, can also bind ligand alone or in combination with the extracellular domain, and are therefore also useful for in vitro ligand-binding assays.

The term “transmembrane domain,” which comprises the seven “transmembrane regions,” refers to the domain of TAS 1R or TAS2R polypeptides that lies within the plasma membrane, and may also include the corresponding cytoplasmic (intracellular) and extracellular loops. In one embodiment, this region corresponds to the domain of TAS 1R or TAS2R family members. In the case of TAS 1R family member this starts approximately at the conserved glutamic acid residue at amino acid position 563 plus or minus 20 amino acids and ends approximately at the conserved tyrosine amino acid residue at position 812 plus or minus approximately 10 amino acids. The seven transmembrane regions and extracellular and cytoplasmic loops can be identified using standard methods, as described in Kyte & Doolittle, J. Mol. Biol., 157:105-32 (1982)) (46), or in Stryer, supra (45).

The term “cytoplasmic domains” refers to the domains of TAS 1R or TAS2R polypeptides that face the inside of the cell, e.g., the “C-terminal domain” and the intracellular loops of the transmembrane domain, e.g., the intracellular loop between transmembrane regions 1 and 2, the intracellular loop between transmembrane regions 3 and 4, and the intracellular loop between transmembrane regions 5 and 6. “C-terminal domain” refers to the region that spans the end of the last transmembrane domain and the 0-terminus of the protein, and which is normally located within the cytoplasm. In one embodiment, this region starts at the conserved tyrosine amino acid residue at position 812 plus or minus approximately 10 amino acids and continues to the C-terminus of the polypeptide.

The term “ligand-binding region” or “ligand-binding domain” refers to sequences derived from a taste receptor, particularly a taste receptor that substantially incorporates at least the extracellular domain of the receptor. In one embodiment, the extracellular domain of the ligand-binding region may include the N-terminal domain and, optionally, portions of the transmembrane domain, such as the extracellular loops of the transmembrane domain. The ligand-binding region may be capable of binding a ligand, and more particularly, a compound that enhances, mimics, blocks, or modulates taste, e.g., sweet, bitter, or umami taste. In the case of TAS2Rs, the compound bound by the ligand binding region will modulate bitter taste. In the case of TAS1Rs, the compound bound by the ligand-binding region will modulate sweet or umami taste.

The phrase “heteromultimer” or “heteromultimeric complex” in the context of the TAS1R receptors or polypeptides used in the assays of the present disclosure refers to a functional association of at least one TAS1R receptor and another receptor, typically another TAS1R receptor polypeptide (or, alternatively another non-TAS1R receptor polypeptide). For clarity, the functional co-dependence of the TAS1Rs is described in this application as reflecting their possible function as heterodimeric taste receptor complexes. However, as discussed in Senomyx patent applications and publications which are incorporated by reference herein, (10-12) functional co-dependence may alternatively reflect an indirect interaction. For example, TAS1R3 may function solely to facilitate surface expression of TAS1R1 and TAS1R2, which may act independently as taste receptors. Alternatively, a functional taste receptor may be comprised solely of TAS1R3, which is differentially processed under the control of TAS1R1 or TAS1R2, analogous to RAMP-dependent processing of the calcium-related receptor. By contrast, in the case of TAS2Rs the eukaryotic cells used in the subject MAPK assays will generally express a single TAS2R.

The phrase “modulator” or “modulatory compound” means any compound that itself affects the activity of a TAS1R or TAS2R or modulates (affects) the effect of another compound on TAS1R or TAS2R activity. Typically, modulation is determined by cell-based assays that detect the effect of a putative modulator or Gi signaling pathways, e.g., assays that detect the effect of a compound on MAPK activity, cAMP levels or adenylyl cyclase activity.

The phrases “functional taste receptor,” “functional receptor”, “functional TAS1R,” “functional TAS2R,” “functional TAS1R2” and the like refer to a polypeptide having the sequence of the identified receptor(s) as well as any fragment, domain, chimera, or sequence variants thereof that bind to a ligand that binds to the wild-type polypeptide. Sequence variants have, in some embodiments, from 80% to 99.5% sequence identity to the wild-type polypeptide, e.g., at least 85%, at least 87.5%, at least 90%, at least 91%, at least 92%, at least 93%, at least 94%, at least 95%, at least 96%, at least 97%, at least 98%, at least 99%, or at least 99.5% identity. Percentage sequence identity is readily determined using sequence alignment methods known in the art, e.g., Smith-Waterman alignment.

The phrase “wild-type” refers to polypeptide and polynucleotide sequences as published in literature and publicly accessible databases as of the date of filing of this application, and encompasses published naturally occurring sequence variants (for example, those published by the HapMap project or available in other publicly accessible sequence databases). An example of wild-type sequences include: rTAS1R1 (Accession # MD18069) (Hoon et al., Cell 96 (4): 541-51 (1999)); rT1R2 (Accession # MD18070) (Hoon et al., Cell 96(4): 541-59 (1999)); mT1R1 (Accession # MK39437); mT1R2 (Accession #AAK 39438); mT1R3 (Accession MK 55537) (Max et al., Nat. Genet. 28(1): 58-63 (2001)); rT1R1 (Accession # AAK07092) (Li et al., Mamm Genome (1 2(1): 13-16 (2001)); mT1R1 (Accession # NP 114073); mT1R1 (Accession # MK07091) (Li et al., Mamm. Genome (121):13-16 (2001)); rT1R2 (Accession # MD18070) (Hoon et al., Cell 9664): 541-551 (1999)); mT1R2 (Accession # NP114079); mT1R3 (Accession # AAK39436); mT1R3 (Accession # BAB47181) (Kitagawa et al., Biochem. Biophys. Res. Comm 283(1):23642 (2001)); mT1R3 (Accession #NP1 14078); mT1R3 (Accession # AAK55536) (Max et al., Nat. Genet. 28(1):58-63 (2001)); mT1R3 (Accession No. AAK01937); and the canine and feline sequences disclosed in U.S. Pat. No. 7,541,158, “Taste receptors of the TAS1R family from domestic dog” and U.S. Pat. No. 7,527,944, “Taste receptors of the TAS1R family from domestic cat.”

The phrases “chimeric gene,” “chimeric polypeptide,” “chimera,” “TAS1R chimera,” “TAS1R chimera” and the like refer to a polypeptide comprising portions of a TAS1R or TAS2R taste receptor gene and a different GPCRs or another species' ortholog of the same GPCR, or a gene encoding such a polypeptide. For example, a domain of a TAS1R polypeptide, e.g., an extracellular, transmembrane, or intracellular domain, may be fused to a heterologous polypeptide, thereby forming a chimeric polypeptide, e.g., a chimeric protein with GPCR activity. In some cases, chimeras comprise complete domains of different GPCRs, such as an extracellular domain of one GPCR and a transmembrane domain of a different GPCR, and an intracellular domain which may be of the same or a different GPCR than the other domains. Chimeric GPCRs comprise, in some cases, domains of different GPCRs of the same family, e.g., a TAS1R and another TAS1R or a different members of GPCR family 3 (metabotropic glutamate receptor, vomeronasal receptor, type 2, extracellular calcium-sensing receptor). Chimeric taste receptors have been reported to retain ligand binding function (see Jiang et al. “The cysteine-rich region of TAS1R3 determines responses to intensely sweet proteins.” J Biol Chem 2004, 279:45068-45075; Xu et al. “Different functional roles of TAS1R subunits in the heteromeric taste receptors.” Proc Natl Acad Sci U S A 2004, 101:14258-14263; Jiang et al. “Identification of the cyclamate interaction site within the transmembrane domain of the human sweet taste receptor subunit TAS1R3.” J Biol Chem 2005, 280:34296-34305; Winnig et al. “The binding site for neohesperidin dihydrochalcone at the human sweet taste receptor.” BMC Struct Biol. 2007 Oct 12;7:66). Such proteins are useful, e.g., in assays to identify ligands, agonists, antagonists, or other modulators of TAS1R receptors. For example, a TAS1R polypeptide can be expressed in a eukaryotic cell as a chimeric receptor with a heterologous, chaperone sequence that facilitates plasma membrane trafficking, or maturation and targeting through the secretory pathway. The optional heterologous sequence may be a PDZ domain-interacting peptide, such as a C-terminal PDZIP fragment. PDZIP is an ER export signal, which, according to the present invention, has been shown to facilitate surface expression of heterologous proteins such as the TAS1R receptors described herein. More particularly, in one aspect of the invention, PDZIP can be used to promote proper targeting of problematic membrane proteins such as olfactory receptors, TAS2R taste receptors, and the TAS1R taste receptors described herein. Such chimeric TAS1R receptors can be expressed in any eukaryotic cell, such as U2OS cells. In some cases, the cells contain a G protein, which may be a promiscuous G protein such as Gα15 or Gα16 or another G protein capable of linking GPCRs to an intracellular signaling pathway or to a signaling protein such as phospholipase C. Activation of such chimeric receptors in such cells can be detected using any standard method, such as by detecting changes in intracellular calcium by detecting FURA-2 dependent fluorescence in the cell, for example, using a cell-based ligand free assay. If host cells do not express an appropriate G protein, they may be transfected with a gene encoding a promiscuous G protein such as those described in U.S. Application Ser. No. 60/243,770, U.S. application Ser. No. 09/984,297, filed Oct. 29, 2001, and U.S. application Ser. No. 09/989,497 filed Nov. 21, 2001 each of which is herein incorporated by reference in its entirety.

The terms “assay,” “assaying” or like terms refers to an analysis to determine, for example, the presence, absence, quantity, extent, kinetics, dynamics, or type of a cell's response upon stimulation with an exogenous stimulus, such as a ligand candidate compound, adenylate activator, cAMP analog, or a GPCR ligand or ligand candidate. In some cases, assays determine a cell's optical or bioimpedance response upon stimulation with an exogenous stimulus.

The terms “attach,” “attachment,” “adhere,” “adhered,” “adherent,” “immobilized,” or like terms generally refer to immobilizing, fixing, culturing, incubating, and like terms, for example, a cell, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials, a compatibilizer (e.g., fibronectin, collagen, lamin, gelatin, polylysine, etc.), or both, and like interactions.

The term “adherent cells” refers to a cell, a cell line, or a cell system, such as a prokaryotic or eukaryotic cell, that remains associated with, immobilized on, or in certain contact with the outer surface of a substrate. Such cells after culturing can withstand or survive washing and medium exchanging process, which is prerequisite to many cell-based assays. “Weakly adherent cells” refers to a cell, a cell line, or a cell system, such as a prokaryotic or eukaryotic cell, which weakly interacts, associates with or contacts the surface of a substrate during cell culture. However, these types of cells, for example, human embryonic kidney cells, tend to dissociate easily from the surface of a substrate by physically disturbing approaches such as washing or medium exchange. “Suspension cells” refers to a cell or a cell line that is cultured in a medium wherein the cells do not attach or adhere to the surface of a substrate during the culture. “Cell culture” or “cell culturing” refer to the process by which either prokaryotic or eukaryotic cells are grown under controlled conditions. “Cell culture” can refer to the culturing of cells derived from multicellular eukaryotes, especially animal cells, and to culturing of complex tissues and organs.

The term “biosensor” or a like term refers to a device for the detection of an analyte (or measurement of a property of an analyte) that combines a biological component with a physicochemical detector component. The biosensor typically consists of three parts: a biological component or element (such as tissue, microorganism, pathogen, cells, or combinations thereof), a detector element (operating in a physicochemical way such as optical, piezoelectric, electrochemical, thermometric, or magnetic), and a transducer associated with both components. The biological component or element can be, for example, a live-cell. In embodiments, an optical biosensor can comprise an optical transducer for converting a molecular recognition or molecular stimulation event in a living-cell into a quantifiable signal.

“Label-free,” “label free” or a liketerms refer to a device (such as a biosensor) or an assay method that can detect an analyte or measure a property of an analyte without requiring the presence of an added label. Though not required, a label may be present, for example as an enhancer of detection or used for another purpose (e.g., a vital dye to assist in monitoring cell viability). Examples of label-free assay systems are described under the heading “Label-Free Biosensor-Based Cell Assays For Detecting Taste Receptor Modulators.”

The phrase “functional effects” in the context of assays for testing compounds that modulate at least one TAS1R or TAS2R family member mediated taste transduction includes the determination of any parameter that is indirectly or directly under the influence of the receptor, e.g., functional, physical and chemical effects. It includes ligand binding, changes in cell mass distribution, cell adhesion, cell morphology, ion flux, membrane potential, current flow, transcription, G protein binding, GPCR phosphorylation or dephosphorylation, conformation change-based assays, signal transduction, receptor-ligand interactions, second messenger concentrations (e.g., cAMP, cGMP, IP3, K+, or Ca2+ ), in vitro, in vivo, and ex vivo and also includes other physiologic effects such increases or decreases of neurotransmitter or hormone release. In the present disclosure, the assays will generally measure the effect of a compound in cell-based expression systems whereby the TAS1R or TAS2R is functionally coupled to a G protein and the assays are used to screen for putative sweeteners or sweet taste modulators or enhancers, umami taste modulators or enhancers, or bitter compounds or bitter taste modulators or enhancers, e.g., bitter taste blockers. Such modulators have application for incorporation in foods, beverages, pharmaceuticals, and the like for human or animal consumption.

By “determining the functional effect” in the context of assays is meant assays for a compound that increases or decreases a parameter that is indirectly or directly under the influence of at least one TAS1R or TAS2R family member, e.g., functional, physical and chemical effects. Such functional effects can be measured by any means known to those skilled in the art, e.g., detecting changes in spectroscopic characteristics (e.g., fluorescence, absorbency, refractive index), hydrodynamic (e.g., shape), changes in cell mass distribution, cell adhesion, cell morphology, resonant waveguide grating (RWG) biosensors (e.g., utilizing optical waveguide grating or impedance-based grating) and electrical biosensors, chromatographic, or solubility properties, patch clamping, voltage-sensitive dyes, whole cell currents, radioisotope efflux, inducible markers, oocyte TAS1R or TAS2R gene expression; tissue culture cell TAS1R or TAS2R expression; transcriptional activation of TAS1R or TAS2R genes; ligand-binding assays; voltages, membrane potential and conductance changes; ion flux assays; changes in intracellular second messengers such as cAMP, cGMP, and inositol triphosphate (IP3); changes in intracellular potassium or calcium levels; neurotransmitter release, conformational assays and the like.

“Inhibitors,” “activators,” “enhancer,” and “modulators” of TAS1R or TAS2R genes or proteins are used to refer to inhibitory, activating, or modulating molecules identified using in vitro and in vivo assays for taste transduction, e.g., ligands, agonists, antagonists, inversed agonists, and their homologues and mimetics. These compounds themselves modulate TAS1R or TAS2R activity or modulate the effect of another compound on TAS1R or TAS2R activity. In some embodiments, the modulators will block taste of a known bitter compound or enhance the taste of a known sweet or umami compound or compounds.

Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize, or up regulate taste transduction, e.g., agonists. Modulators include compounds that, e.g., alter the interaction of a receptor with: extracellular proteins that bind activators or inhibitor (e.g., ebnerin and other members of the hydrophobic carrier family); G proteins; kinases (e.g., homologues of rhodopsin kinase and beta adrenergic receptor kinases that are involved in deactivation and desensitization of a receptor); and arrestins, which also deactivate and desensitize receptors. Modulators can include genetically modified versions of TAS1R or TAS2R family members, e.g., with altered activity, as well as naturally occurring and synthetic ligands, antagonists, agonists, small chemical molecules and the like. Such assays for inhibitors and activators include, e.g., expressing TAS1R or TAS2R family members in cells or cell membranes, applying putative modulator compounds, in the presence or absence of tastants, e.g., sweet, umami or bitter tastants, and then determining the functional effects on taste transduction, as described above. Samples or assays comprising TAS1R or TAS2R family members that are treated with a potential activator, inhibitor, or modulator are compared to control samples without the inhibitor, activator, or modulator to examine the extent of modulation. Positive control samples (e.g. a sweet, umami, or bitter tastant without added modulators) are assigned a relative TAS1R or TAS2R activity value of 100%.

Negative control samples (e.g., buffer without an added taste stimulus) are assigned a relative TAS1R or TAS2R activity value of 0%. Inhibition of a TAS1R or TAS2R is achieved when a mixture of the positive control sample and a modulator result in the TAS1R or TAS2R activity value relative to the positive control is 80%, optionally 50% or 25-0%. Activation of a TAS1R or TAS2R by a modulator alone is achieved when the TAS1R activity value relative to the positive control sample is 10%, 25%, 50%, 75%, optionally 100%, optionally 150%, optionally 200-500%, or 1000-3000% higher.

The terms “purified,” “substantially purified,” and “isolated” as used herein refer to the state of being free of other, dissimilar compounds with which the compound is normally associated in its natural state, so that the “purified,” “substantially purified,” and “isolated” subject comprises at least 0.5%, 1%, 5%, 10%, or 20%, and, in some embodiments, at least 50% or 75% of the mass, by weight, of a given sample. In some embodiments, these terms refer to the compound comprising at least 95% of the mass, by weight, of a given sample. As used herein, the terms “purified,” “substantially purified,” and “isolated,” when referring to a nucleic acid or protein, also refers to a state of purification or concentration different than that which occurs naturally in the mammalian, especially human body. Any degree of purification or concentration greater than that which occurs naturally in the mammalian, especially human, body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones (see e.g., Oligonucleotides and Analogues, a Practical Approach, ed. F. Eckstein, Oxford Univ. Press (1991); Antisense Strategies, Annals of the N.Y. Academy of Sciences, Vol. 600, Eds. Baserga et al. (NYAS 1992); Milligan J. Med. Chem. 36:1923-1937 (1993); Antisense Research and Applications (1993, CRC Press), Mata, Toxicol. Appl. Pharmacol. 144:189-197 (1997); Strauss-Soukup, Biochemistry 36:8692-8698 (1997); Samstag, Antisense Nucleic Acid Drug Dev, 6:153-156 (1996)) (47-53).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base or deoxyinosine residues (Batzer et al, Nucleic Acid Res., 19:5081 (1991); Ohtsuka et al., J. Biol. Chem., 260:2605-2608 (1985); Rossolini et al., Mol. Cell. Probes, 8:91-98 (1994)) (54-56). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “plasma membrane translocation domain” or simply “translocation domain” means a polypeptide domain that, when incorporated into a polypeptide coding sequence, can with greater efficiency “chaperone” or “translocate” the hybrid (“fusion”) protein to the cell plasma membrane than without the domain. For instance, a “translocation domain” may be derived from the amino terminus of the bovine rhodopsin receptor polypeptide, a 7-transmembrane receptor. However, rhodopsin from any mammal may be used, as can other translocation facilitating sequences. Thus, the translocation domain is particularly efficient in translocating 7-transmembrane fusion proteins to the plasma membrane, and a protein (e.g., a taste receptor polypeptide) comprising an amino terminal translocating domain will be transported to the plasma membrane more efficiently than without the domain. However, if the N-terminal domain of the polypeptide is active in binding to some ligands, as with examples of TAS1R or TAS2R receptors, the use of other translocation domains may be possible. For instance, a PDZ domain-interacting peptide, as described herein, may be used.

The “translocation domain,” “ligand-binding domain,” and chimeric receptors compositions described herein also include “analogs,” or “conservative variants” and “mimetics” (“peptidomimetics”) with structures and activity that substantially correspond to the certain sequences. Thus, the terms “conservative variant” or “analog” or “mimetic” refer to a polypeptide which has a modified amino acid sequence, such that the change(s) do not substantially alter the polypeptide's (the conservative variant's) structure or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide, is implicit in each described sequence.

Conservative substitution tables providing functionally similar amino acids are well known in the art. For example, one example of a guideline to select conservative substitutions includes (original residue followed by substitution): ala/gly or ser; arg/lys; asn/gln or his; asp/glu; cys/ser; gln/asn; gly/asp; gly/ala or pro; his/asn or gin; ile/leu or val; leu/ile or val; lys/arg or gln or glu; met/leu or tyr or lie; phe/met or leu or tyr; ser/thr; thr/ser; trp/tyr; tyr/trp or phe; val/ile or leu. An alternative guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (T), Leucine (L), Methionine (M), Valine (V); and 6) Phenylalanine (F), Tyrosine (Y), Tryptophan (W); (see also, e.g., Creighton, Proteins, W.H. Freeman and Company (1984); Schultz and Schimer, Principles of Protein Structure, Springer-Verlag (1979)) (57-58). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The term “mimetic” and “peptidomimetic” refer to a synthetic chemical compound that has substantially the same structural or functional characteristics of the polypeptides, e.g., translocation domains, ligand-binding domains, or chimeric receptors. The mimetic can be either entirely composed of synthetic, non-natural analogs of amino acids, or may be chimeric molecules of partly natural peptide amino acids and partly non-natural analogs of amino acids. The mimetic can also incorporate any amount of natural amino acid conservative substitutions as long as such substitutions also do not substantially alter the mimetic's structure or activity.

As with polypeptides which are conservative variants, routine experimentation will determine whether a mimetic's structure or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(0)—CH₂— and —C(O)—NH—), aminomethylene (CH₂—NH), ethylene, olefin (CH—CH), ether (CH₂—O), thioether (CH₂—S), tetrazole (CN₄), thiazole, retroamide, thioamide, or ester (see, e.g., Spatola, Chemistry and Biochemistry of Amino Acids, Peptides and Proteins, Vol. 7, pp 267-357, “Peptide Backbone Modifications,” Marcell Dekker, NY (1983)) (157). A polypeptide can also be characterized as a mimetic by containing all or some non-natural residues in place of naturally occurring amino acid residues; non-natural residues are well described in the scientific and patent literature.

A “label” or a “detectable moiety” is a composition detectable by spectroscopic, photochemical, biochemical, immunochemical, or chemical means. For example, useful labels include 32 P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogene, or indirectly labeled such as with biotin to which a streptavidin complex may later bind. By assaying for the presence or absence of the probe, one can detect the presence or absence of the select sequence or subsequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions.

An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain and a nucleic acid sequence amplified using a primer.

As used herein, a “stable cell line” refers to a cell line, which stably, i.e. over a prolonged period, expresses a heterologous nucleic sequence, i.e., a TAS1R, TAS2R or G protein. In certain embodiments, such stable cell lines will be produced by transfecting appropriate cells, typically mammalian cells, with a linearized vector that contains a TAS1R or TAS2R expression construct that expresses at least one TAS1R or TAS2R, i.e., TAS1R1, TAS1R2 or TAS1R3 or a TAS2R. In some cases, such stable cell lines that express a functional TAS1R or TAS2R receptor will be produced by co-transfecting two linearized plasmids that express hT1R1 and hT1R3 or hT1R2 and hT1R3 or a single line plasmid that expresses a specific TAS2R and an appropriate selection procedure to generate cell lines having these genes stably integrated therein.The term “expression vector” refers to any recombinant expression system for the purpose of expressing a nucleic acid sequence in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes” which contain only the minimum elements needed for transcription of the recombinant nucleic acid.

By “host cell” is meant a cell that contains an expression vector and supports the replication or expression of the expression vector. Host cells may be prokaryotic cells such as E. coli, or eukaryotic cells such as yeast, insect, amphibian, worm or mammalian cells such as CHO, Hela, BHK, U2OS cells, and the like, e.g., cultured cells, explants, and cells in vivo.

The term “substantially identical”, is used herein to describe a degree of similarity between nucleotide or polypeptide sequences, and refers to two or more sequences that have at least least 60%, at least 70%, at least 80%, 90% to 99%, still 95% to 99%, or 99% nucleotide or polypeptide identify, when compared and aligned for maximum correspondence, as measured using one of the following sequence comparison algorithms or by visual inspection. In some embodiments, the substantial identity exists in nucleotide or polypeptide sequences of at least 100 residues, or at least 150 residues, or a full length coding sequence. The term “full length” is used herein to refer to a complete open reading frame encoding a functional TAS1R or TAS2R polypeptide, as described further herein below. Methods for determining percent identity between two polypeptides are defined herein below under the heading “Nucleotide and Amino Acid Sequence Comparisons.”

In one aspect, substantially identical sequences can be polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise one or more residue changes, a deletion of residues, or an insertion of additional residues.

Another indication that two nucleotide sequences are substantially identical is that the two molecules hybridize specifically to or hybridize substantially to each other under stringent conditions. In the context of nucleic acid hybridization, two nucleic acid sequences being compared can be designated a “probe” and a “target.” A “probe” is a reference nucleic acid molecule, and a “target” is a test nucleic acid molecule, often found within a heterogeneous population of nucleic acid molecules. A “target sequence” is synonymous with a “test sequence.”

In some cases, a nucleotide or polypeptide sequence employed for hybridization studies or assays includes probe sequences that are complementary to or mimic at least an 14 to 40 nucleotide sequence of a nucleic acid molecule. In some cases, probes comprise 14 to 20 nucleotides, or even longer where desired, such as 30, 40, 50, 60, 100, 200, 300, or 500 nucleotides or up to the full length of the particular TAS1R or TAS2R. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” and “stringent hybridization wash conditions” refer to conditions under which a probe will hybridize to its target subsequence, typically in a complex mixture of nucleic acids but to no other sequences. Stringent conditions are sequence-dependent and will be different in different circumstances. Longer sequences hybridize specifically at higher temperatures. An extensive guide to the hybridization of nucleic acids is that in Tigssen, Techniques in Biochemistry and Molecular Biology—Hybridization With Nucleic Probes, “Overview of principles of hybridization and the strategy of nucleic acid assays.” (1973) Generally, highly stringent hybridization and wash conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium).

Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the additional of destablizing agents such as formamide For selective or specific hybridization, a positive signal is at least two times background, optionally 10 times background hybridization. Examples of stringent hybridization conditions are: 50% formamide, 5×SSC, and 1% SDS, incubating at 42° C. or 5×SSC, 1% SDS, incubating at 65° C. The hybridization and wash steps effected in said stringent hybridization conditions are each effected for at least 1, 2, 5, 10, 15, 30, 60, or more minutes. In some cases, the wash and hybridization steps are each effected for at least 5 minutes, or at least 10 minutes, or at least 15 minutes.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

An example of stringent hybridization conditions for Southern or Northern Blot analysis of complementary nucleic acids having more than 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. (60) for a description of SSC buffer. Often, a high stringency wash is preceded by a low stringency wash to remove background probe signal. An example of medium stringency wash conditions for a duplex of more than 100 nucleotides, is 15 minutes in 1×SSC at 45° C. An example of low stringency wash for a duplex of more than 100 nucleotides, is 15 minutes in 4× to 6×SSC at 40° C. For short probes (e.g., 10 to 50 nucleotides), stringent conditions typically involve salt concentrations of less than about 1 M Na+ ion, typically about 0.01 to 1 M Na+ion concentration (or other salts) at pH 7.0-8.3, and the temperature is typically at least about 30° C. Stringent conditions can also be achieved with the addition of destablizing agents such as formamide In general, a signal to noise ratio of 2-fold (or higher) than that observed for an unrelated probe in the particular hybridization assay indicates detection of a specific hybridization.

The following are additional examples of hybridization and wash conditions that can be used to identify nucleotide sequences that are substantially identical to reference nucleotide sequences: a probe nucleotide sequence, in some cases, hybridizes to a target nucleotide sequence in 7% sodium dodecyl sulfate (SDS), 0.5M NaPO 4 , 1 mM EDTA at 50° C. followed by washing in 2xSSC, 0.1% SDS at 50° C.; or a probe and target sequence hybridize in 7% sodium dodecyl sulfate (SDS), 0.5M NaPO4, 1 mM EDTA at 50° C. followed by washing in 1×SSC, 0.1% SDS at 50° C.; or a probe and target sequence hybridize in 7% sodium dodecyl sulfate (SDS), 0.5M Na3PO4, 1 MM EDTA at 50° C. followed by washing in 0.5xSSC, 0.1% SDS at 50° C.; or a probe and target sequence hybridize in 7% sodium dodecyl sulfate (SIDS), 0.5M NaPO 4 , 1 mM EDTA at 50° C. followed by washing in 0.1×SSC, 0.1 SDS at 50° C.; or a probe and target sequence hybridize in 7% sodium dodecyl sulphate (SDS), 0.5M NaPO 4 , 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. Nucleic acid molecules that do not hybridize to each other under stringent conditions are still substantially identical if the corresponding proteins are substantially identical. This can occur, for example, when two nucleotide sequences comprise conservatively substituted variants as permitted by the genetic code.

Nucleic acids that do not hybridize to each other under stringent conditions are still substantially related if the polypeptides that they encode are substantially related. This occurs, for example, when a copy of a nucleic acid is created using the maximum codon degeneracy permitted by the genetic code. In such cases, the nucleic acids typically hybridize under moderately stringent hybridization conditions. Examples of “moderately stringent hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. In some cases, the wash and hybridization steps are each effected for at least 5 minutes. A positive hybridization is at least twice background. Those of ordinary skill will readily recognize that alternative hybridization and wash conditions can be utilized to provide conditions of similar stringency.

The term “conservatively substituted variants” refers to nucleic acid sequences having degenerate codon substitutions wherein the third position of one or more selected (or all) codons is substituted with mixed-base or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98 (54-56).

The term “TAS1R or TAS2R” also encompasses nucleic acids comprising subsequences and elongated sequences of a TAS1R or TAS2R nucleic acid, including nucleic acids complementary to a TAS1R or TAS2R nucleic acid, TAS1R or TAS2R RNA molecules, and nucleic acids complementary to TAS1R or TAS2R RNAs (cRNAs).

The term “subsequence” refers to a sequence of nucleic acids that comprises a part of a longer nucleic acid or polypeptide sequence. An example of a subsequence is a probe, described herein above, or a primer. The term “primer” as used herein refers to a contiguous sequence comprising 8 or more deoxyribonucleotides or ribonucleotides, or 10-20 nucleotides, or 20-30 nucleotides of a selected nucleic acid molecule. The primers encompass oligonucleotides or polypeptides of sufficient length and appropriate sequence so as to provide initiation of polymerization on a nucleic acid molecule.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. I n addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences,” as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “operatively linked,” as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a TAS1R or TAS2R polypeptide, as described further herein below.

The term “construct,” as used herein to describe a type of construct comprising an expression construct, refers to a vector further comprising a nucleotide sequence operatively inserted with the vector, such that the nucleotide sequence is recombinantly expressed.

The terms “recombinantly expressed” or “recombinantly produced” are used interchangeably to refer generally to the process by which a polypeptide encoded by a recombinant nucleic acid is produced.

The term “heterologous nucleic acids” refers to a sequence that originates from a source foreign to an intended host cell or, if from the same source, is modified from its original form. Thus, in some cases, recombinant TAS1R or TAS2R nucleic acids comprise heterologous nucleic acids. A heterologous nucleic acid in a host cell can comprise a nucleic acid that is endogenous to the particular host cell but has been modified, for example by mutagenesis or by isolation from native cis-regulatory sequences. A heterologous nucleic acid also includes non-naturally occurring multiple copies of a native nucleotide sequence. A heterologous nucleic acid can also comprise a nucleic acid that is incorporated into a host cell's nucleic acids at a position wherein such nucleic acids are not ordinarily found.

Nucleic acids used in the cell-based assays can be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) Molecular Cloning: A Laboratory Manual. Cold Spring Harbor Laboratory Press, Cold Spring Harbor, N.Y. (1989); Silhavy et al. Experiments with Gene Fusions. Cold Spring Harbor Laboratory Press, Cold Spring Harbor, N.Y. (1984); Glover & Hames DNA Cloning: A Practical Approach, 2nd ed. IRL Press and Oxford University Press, Oxford, N.Y. (1995); Ausubel (ed.) Short Protocols in Molecular Biology, 3rd ed. Wiley, N.Y. (1995).

The term “functionally equivalent” in the context of amino acids is known in the art and is based on the relative similarity of the amino acid side-chain substituents. See Henikoff & Henikoff Adv. Protein Chem 54:73-97 (2000). Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., J. Mol. Biol. 157(1):105-32 (1982)) (69). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The assays of the present disclosure also can use functional fragments of a particular TAS1R or TAS2R polypeptide. Such functional portion need not comprise all or substantially all of the amino acid sequence of a native TAS1R or TAS2R gene product. The assays of the present disclosure also can use functional polypeptide sequences that are longer sequences than that of a native TAS1R or TAS2R polypeptide. For example, one or more amino acids can be added to the N-terminus or C-terminus of a TAS1R or TAS2R polypeptide. Such additional amino acids can be employed in a variety of applications, including but not limited to purification applications. Methods of preparing elongated proteins are known in the art.

The term “promoter,” as used herein, refers to the transcriptional control elements necessary for receptor-mediated regulation of gene expression, including not only the basal promoter, but also any enhancers or transcription-factor binding sites necessary for receptor-regulated expression, such as woodchuck post-translational regulatory element (“WPRE”).

Other terms and general information are set forth in other portions of this description, even though not included in this subsection.

### Methods and Assays

The present disclosure describes a new screening approach to identify taste receptor modulators. This approach relies on the capacity of GPCRs to cause simultaneous activation of several G protein dependent or G protein-independent signaling pathways. Emerging technologies allow the measurement and integration of these activated pathways quantitatively into one read-out even without using cells molecularly engineered for forced or promiscuous G protein coupling. Recently available optical biosensors such as the SRU BIND, CELL KEY, ECIS, EPIC, RT-CES, and XCELLIGENCE use optical waveguide grating or impedance-based grating to precisely quantify GPCR-induced changes in cell mass distribution or cell adhesion and cell morphology, respectively, at a focal point near the substratum. Kinetics of DMR (dynamic mass redistribution) or changes in impedance have shown that Gi, Gq, Gs, G12/G13-coupled receptors exhibit very specific signatures or waveforms, that are cell type dependent and that allow investigators to not only measure modulator responses but also to deconvolute which pathway is activated in the cells.

The human sweet taste receptor, human umami taste receptor, and bitter taste receptors each functionally couple to G proteins, such as Gi/o proteins, to transmit intracellular signals and to gustducin and G14 expressed in taste receptor cells. Taste receptor cells from the tongue have not been reported to be efficiently grown in the lab. Other cell types expressing taste receptors have been detected. For example, cells along the gastrointestinal tract and the pancreas expressing sweet taste receptor subunits and gustducin have been detected. In addition, Gi/o proteins are ubiquitously expressed in numerous cell types including but not limited to CRL-1573, U2OS, CHO-K1, COS-7, Swiss 3T3.

In one embodiment, the genes for TAS1R2 and TAS1R3 subunits are delivered transiently to cells or a stable cell line expressing the sweet taste receptor without overexpressed G proteins is made. Typical cells for such an experiment would be but are not limited to:

CRL-1573, U205, CHO-K1, COS-7, Swiss 3T3. Cells are then plated onto proprietary biosensor 384-well plates from Corning and the DMR responses are measured after stimulation. In a second embodiment, cells endogenously expressing TAS1R subunits with or without endogenous expression of gustducin are used for such an experiment. As demonstrated in the examples below, cells endogenously expressing a TAS1R subunit (with or without gustducin) include cultures of the cell lines: Panc-1, ASPC-1, BxPc-3, HuTu-80, HCT-8, HT-29, SW48, COLO205, COLO320-DM, HCT-15, NCI-H716, CCD841 CoN, CCD-112 CoN, CCD-33Co, Caco-2, Caco2/TC7, STC-1, GluTAG, N38, and MIN6. In addition, other cells, human or non-human primate cells may be detected that endogenously express a functional TAS1R or TAS2R receptor and used in the subject assays. The invention however contemplates the use of any cell that endogenously expresses a TAS1R or TAS2R including rare cells (cells that do not typically express TAS1Rs or TAS2Rs endogenously (for example, derived from a population of which less than 1-5% of cells endogenously express said TAS1R or TAS2R)).

Examples of such human or other mammalian cells for use in the subject methods include: exocrine secretory epithelial cells such as salivary mucous cells, salivary serous cells, Von Ebner's gland cells, mammary gland cells, lacrimal gland cells, ceruminous gland cells, eccrine sweat gland dark cells, eccrine sweat gland clear cells, apocrine sweat gland cells, gland of Moll cell in eyelid, sebaceous gland cells, Bowman's gland cells, Brunner's gland cells, seminal vesicle cells, prostate gland cells, Bulbourethral gland cells, Gland of Littre cells, Uterus endometrium cells, isolated goblet cells of respiratory and digestive tracts, stomach lining mucous cells, Gastric gland zymogenic cells, gastric gland oxyntic cells, pancreatic acinar cells, Paneth cells of small intestine, type II pneumocytes of lung, Clara cells of lung; hormone secreting cells such as anterior pituitary cells, somatotropes, lactotropes, Thyrotropes, Gonadotropes, Corticotropes, intermediate pituitary cell, secreting melanocyte-stimulating hormone cell, magnocellular neurosecretory cells, oxytocin secreting cells, vasopressin secreting cells, gut and respiratory tract cells secreting serotonin, endorphin, somatostatin, gastrin, secretin, cholecystokinin, insulin, glucagon, or bombesin, thyroid gland cells, thyroid epithelial cells, parafollicular cells, parathyroid gland cells, parathyroid chief cells, oxyphil cells, adrenal gland cells, chromaffin cells, cells secreting steroid hormones, Leydig cell of testes secreting testosterone, Theca interna cells of ovarian follicle secreting estrogen, Corpus luteum cell of ruptured ovarian follicle progesterone, Granulosa lutein cells, Theca lutein cells, Juxtaglomerular cells, Macula densa cells of kidney, Peripolar cell of kidney, Mesangial cells of kidney, Epithelial cells lining closed internal body cavities such as blood vessel and lymphatic vascular endothelial fenestrated cells, Blood vessel and lymphatic vascular endothelial continuous cells, blood vessel and lymphatic vascular endothelial splenic cells, synovial cells lining joint cavities, hyaluronic acid secreting cells, serosal cells, squamous cells, columnar cell of endolymphatic sac (lining peritoneal, pleural, and pericardial cavities and endolymphatic space of ear, columnar cell of endolymphatic sac, Dark cells (lining endolymphatic space of ear), Vestibular membrane cell (lining endolymphatic space of ear), stria vascularis basal cells (lining endolymphatic space of ear), stria vascularis marginal cell (lining endolymphatic space of ear), Cell of Claudius (lining endolymphatic space of ear), Cell of Boettcher (lining endolymphatic space of ear), Choroid plexus cells, Pia-arachnoid squamous cells, pigmented and non-pigmented ciliary epithelium cells of eye, corneal endothelial cells, Peg cells, ciliated cells with propulsive function such as respiratory tract ciliated cells, Oviduct ciliated cells, uterine endometrial ciliated cells, Rete testis ciliated cells, ductulus efferens ciliated cells, ciliated ependymal cell of central nervous system, keratinizing epithelial cells such as epidermal keratinocytes, epidermal basal cells, keratinocyte of fingernails and toenails, Nail bed basal cells, Medullary hair shaft cells, Cortical hair shaft cells, Cuticular hair shaft cells, Cuticular hair root sheath cells, hair root sheath cell of Huxley's layer, hair root sheath cell of Henle's layer, external hair root sheath cells, hair matrix cells, wet stratified barrier epithelial cells such as surrface epithelial cell of stratified squamous epithelium of cornea, tongue, oral cavity, esophagus, anal canal, distal urethra and vagina, basal cells in epithelia of cornea, tongue, oral cavity, esophagus, anal canal, distal urethra and vagina, urinary epithelium cells lining urinary bladder and urinary ducts, nervous system cells such as sensory transducer cells, Auditory inner hair cell of organ of Corti, auditory outer hair cell of organ of Corti, basal cell of olfactory epithelium, cold-sensitive primary sensory neurons, heat-sensitive primary sensory neurons, Merkel cell of epidermis, Olfactory receptor neurons, pain-sensitive primary sensory neurons, photoreceptor cells of retina, photoreceptor rod cells, photoreceptor sensitive cone cells, proprioceptive primary sensory neurons, touch-sensitive primary sensory neurons, type I carotid body cells, type II carotid body cells, Type I hair cell of vestibular apparatus of ear, Type II hair cell of vestibular apparatus of ear (acceleration and gravity), Type I taste bud cells, autonomic neuron cells, cholinergic neural cells, adrenergic neural cells, Peptidergic neural cells, sense organ and peripheral neuron supporting cells such as Inner pillar cells, outer pillar cells, inner phalangeal cells, outer phalangeal cells, border cells, and Hensen cell of organ of Corti, vestibular apparatus supporting cells, taste bud supporting cells, olfactory epithelium supporting cells, Schwann cells, satellite cells encapsulating peripheral nerve cell bodies, enteric glial cells, central nervous system neurons and glial cells, astrocytes, neuron cells, oligodendrocytes, spindle neurons, lens cells, anterior lens epithelial cells, crystallin-containing lens fiber cells, metabolism and storage cells such as hepatocytes, adipocytes, white fat cells, Brown fat cells, liver lipocytes, barrier function cells (Lung, Gut, Exocrine Glands and Urogenital Tract), kidney cells such as Kidney glomerulus parietal cells, kidney glomerulus podocytes, Kidney proximal tubule brush border cells, Loop of Henle thin segment cells, Kidney distal tubule cells, Kidney collecting duct cells, type I pneumocytes, Pancreatic duct cells (centroacinar cells), Nonstriated duct cells (of sweat gland, salivary gland, mammary gland, etc.), principal cells, intercalated cells, duct cells (of seminal vesicle, prostate gland, etc.), Intestinal brush border cells, exocrine gland striated duct cells, gall bladder epithelial cells, Ductulus efferens nonciliated cells, epididymal principal cells, Epididymal basal cells, extracellular matrix cells, ameloblast epithelial cells, planum semilunatum epithelial cells of vestibular apparatus of ear, Organ of Corti interdental epithelial cells, loose connective tissue fibroblasts, corneal fibroblasts, tendon fibroblasts, bone marrow reticular tissue fibroblasts, other nonepithelial fibroblasts, Pericytes, Nucleus pulposus cells of intervertebral disc, cementoblasts, cementocytes, odontoblasts, odontocytes, Hyaline cartilage chondrocytes, fibrocartilage chondrocytes, elastic cartilage chondrocytes, osteoblasts, osteocytes, Osteoprogenitor cells, hyalocytes of vitreous body of eye, Stellate cells of perilymphatic space of ear, Hepatic stellate cell, Pancreatic stelle cells, Contractile cells, skeletal muscle cells, Red skeletal muscle cells, White skeletal muscle cells, Intermediate skeletal muscle cells, nuclear bag cells of muscle spindle , nuclear chain cell of muscle spindle, satellite cells, heart muscle cells, ordinary heart muscle cells, Nodal heart muscle cells, Purkinje fiber cells, Smooth muscle cells, Myoepithelial cell of iris, Myoepithelial cell of exocrine glands, blood and immune system cells such as erythrocytes, Megakaryocytes, Monocytes, connective tissue macrophages, epidermal Langerhans cells, Osteoclasts, dendritic cells, microglial cells, eosinophil granulocytes, basophil granulocytes, mast cells, Helper T cells, suppressor T cells, cytotoxic T cells, natural killer T cells, B cells, natural killer cells, reticulocytes, stem cells and committed progenitors for the blood and immune system, pigment cells such as melanocytes, retinal pigmented epithelial cells, germ cells, Oogonium/Oocytes, spermatids, spermatocytes, spermatogonium cells, spermatozoon cells, nurse cells, ovarian follicle cells, Sertoli cells, thymus epithelial cells, interstitial cells, and interstitial kidney cells, among others.

The present disclosure provides screening assays for identification of taste receptor modulators. This allows for new assays for identifying compounds that modulate, e.g., enhance agonize or antagonize, the activity of specific TAS1R or TAS2R taste receptors or that modulate the effect of another TAS1R or TAS2R activator compound. Because the assayed cells need not be molecularly engineered for forced or promiscuous G protein coupling, it is expected that “false positive” hits (i.e., compounds that modulate the taste receptor in an in vitro assay but are determined to have unsatisfactory modulatory activity in secondary assays or in vivo) may be decreased when using the subject methods. It is also expected that false-positive hits identified using other methods can be identified as such using the subject methods.

In certain aspects, the disclosure provides a cell-based method of detecting a taste receptor modulating compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with a test compound that potentially modulates the activity of the taste receptor; (c) measuring a functional effect of the test compound on the eukaryotic cell using an assay method that comprises using a biosensor to detect or measure a change in cell mass distribution, impedance, or a combination thereof; and (d) identifying the test compound as a taste receptor modulator based on the functional effect.

In certain aspects, the disclosure provides a cell-based method of detecting a taste receptor modulating compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with a test compound that potentially modulates the activity of the taste receptor; (c) measuring a functional effect of the test compound on the eukaryotic cell using an assay method that comprises detecting or measuring a change in GPCR activity, wherein the change in GPCR activity is not indicated through the use of a label or a dye; and (d) identifying the test compound as a taste receptor modulator based on the functional effect.

In certain aspects, the disclosure provides a cell-based method of detecting a taste receptor modulator compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with an identified taste receptor modulator and a test compound that potentially modulates an effect of the identified taste receptor modulator on activity of the taste receptor; (c) measuring a functional effect of the test compound on the eukaryotic cell using an assay method that comprises using a biosensor to detect or measure a change in cell mass distribution, impedance, or a combination thereof; and (d) identifying the test compound as a taste receptor modulator based on the functional effect.

In certain aspects, the disclosure provides a cell-based method of detecting a taste receptor modulator compound, the method comprising: (a) providing a eukaryotic cell that expresses a taste receptor, wherein the taste receptor comprises a G-coupled protein receptor (GPCR) selected from the group consisting of TAS1R proteins, TAS2R proteins, and combinations thereof; (b) contacting the eukaryotic cell with an identified taste receptor modulator and a test compound that potentially modulates an effect of the identified taste receptor modulator on activity of the taste receptor; (c) measuring a functional effect of the test compound on the eukaryotic cell using an assay method comprising detecting or measuring a change in GPCR activity, wherein the change in GPCR activity is not indicated through the use of a label or a dye; and (d) identifying the test compound as a taste receptor modulator based on the functional effect.

In some embodiments of any of the foregoing aspects, the comestible composition is not a naturally occurring composition.

In some embodiments of any of the foregoing aspects, the assays use optical waveguide grating or impedance-based grating to precisely quantify GPCR-induced changes in cell mass distribution or cell adhesion and cell morphology, respectively, at a focal point near the substratum (examples of such assay systems are described in greater detail below). In some embodiments, the assay systems are used with individual cells or cell populations. Kinetics of DMR (dynamic mass redistribution) or changes in impedance have shown that Gi, Gq, Gs, and G12/13-coupled receptors exhibit very specific signatures or waveforms, that are cell type dependent and that allow investigators to not only measure modulator responses but also to deconvolute which pathway is activated in the cells.

Suitable assay systems include, but are not limited to, optical biosensor such as the SRU BIND, CELL KEY, ECIS, EPIC, RT-CES, and XCELLIGENCE. Further non-limiting examples of assay systems that may be used to detect taste receptor activation are disclosed in the following publications: US 2009/0093011; US 2009/0226931; US 2010/0087332; US 2009/0181409; US 2009/0061416; US 2009/0325211; US 2009/0093013; US 2009/0142790; WO 2006/108183; Fang, Y., et al., Biophys. J, 91, 1925-1940 (2006); Fang, Y., et al., J. Pharmacol. Toxicol. Methods, 55, 314-322 (2007); Fang, Y., Assays and Drug development Technologies. 4: 583-595 (2006); Schroder, R. et al., Nat Biotechnol. (2010) Sep;28(9):943-9; Fang et al., Comb. Chem. High Throughput Screen. (2008) Jun;11(5), 357-69.

The TAS1R and TAS2R taste receptors are G protein-coupled receptors (GPCRs), which are the largest family of cell membrane receptors in the human genome and the richest class of drug targets. The primary function of GPCRs as signaling molecules is to transduce exogenous information into intracellular signals. In classical models of drug action, the primary event is binding of a ligand to its receptor, which, in turn, leads to a cellular effect whose magnitude depends on the intrinsic efficacy of the ligand. Classical receptor-occupancy theory defines the efficacy of ligands as their ability to alter the equilibrium between inactive and active states of the receptor, assuming that all GPCR activities are correlated. But recent evidence suggests that GPCR signaling is highly complex and sophisticated. For example, a receptor may couple simultaneously to more than one G protein subtype, and interact with other signaling molecules and scaffolding proteins such as arrestins. In many instances the activation of a receptor can mediate both G protein-dependent and independent signaling, often in a ligand-dependent manner As a result, GPCRs display rich behaviors in cell systems, and many ligands can induce operative bias to favor specific portions of the cell machinery and exhibit pathway-biased activities and efficacies. The complexity of G protein coupled receptor (GPCR) signaling and ligand-directed functional selectivity calls for high resolution tools for studying GPCR behavior.

In the methods disclosed herein, optical biosensors measure ligand-induced cellular responses of cells by detecting minute changes in local mass density or mass redistribution in the cells. Thus, such optical biosensor-based live-cell assays do not require prior knowledge of cell signaling, and can enable direct measurement of ligand-induced receptor activation and signaling.

A ligand-induced dynamic mass redistribution (DMR) signal measured with an optical biosensor provides a method to study ligand-directed trafficking of receptor signaling (i.e., ligand-selective signaling, or ligand-directed functional selectivity). The DMR can approximately globally represent receptor signaling, provide real time kinetic information, and have sensitivity to signaling pathways. Beside its dynamics and signal amplitudes, the kinetic parameters of a ligand-induced DMR signal provide for analyzing receptor signaling and ligand-biased potencies and efficacies.

Label-free cell-based assays generally employ a biosensor to monitor ligand-induced responses in living cells. A biosensor typically utilizes a transducer such as an optical, electrical, calorimetric, acoustic, or magnetic transducer, to convert a molecular recognition event or a ligand-induced change in a cell layer into a quantifiable signal. These label-free biosensors are commonly used for molecular interaction analysis, which involves characterizing how molecular complexes form and disassociate over time. There are two predominant types of biosensors that can be used for label-free cell-based assays-resonant waveguide grating (RWG) biosensors (e.g., utilizing optical waveguide grating or impedance-based grating) and electrical biosensors, which are further described below.

An RWG biosensor consists of a substrate (e.g., glass), a waveguide thin film with an embedded grating structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent and decays exponentially from the sensor surface; the distance at which it decays to 1/e of its initial value is known as the penetration depth and is a function of the design of a particular RWG biosensor, but is typically on the order of 200 nm. This type of biosensor exploits evanescent waves to characterize ligand-induced alterations of a cell or cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion, status, etc.), which is in direct contact with the surface of the biosensor. When a ligand or an analyte interacts with a cellular target (e.g., a GPCR, a kinase, etc.) in living cells, any change in local refractive index within the cell layer can be detected as a shift in resonant angle (or wavelength).

The Corning EPIC system uses RWG biosensors for label-free biochemical or cell-based assays (Corning Inc., Corning, N.Y.). The EPIC System consists of an RWG plate reader and SBS standard microtiter plates. The detector system in the plate reader exploits integrated fiber optics to measure the shift in wavelength of the incident light as a result of ligand-induced changes in the cells. A series of illumination/detection heads are arranged in a linear fashion so that reflection spectra are collected simultaneously from each well within a column of a 384-well microplate. The whole plate can be scanned so that each sensor can be addressed multiple times, and each column is addressed in sequence. The wavelengths of the incident light are collected and used for analysis. A temperature-controlling unit can be built in the instrument to minimize spurious shifts in the incident wavelength due to the temperature fluctuations.

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. In some cases, a small constant voltage at a fixed frequency or varied frequencies is applied to the electrode or electrode array, and the electrical current through the circuit is monitored over time. The ligand-induced change in electrical current provides a measure of cell response. Impedance whole-cell sensing measurements were first achieved in 1984. Since then impedance-based measurements have been applied to study a wide range of cellular events, including cell adhesion and spreading, cell micromotion, cell morphological changes, and cell death. Such impedance systems suffer from high assay variability due to use of a small detection electrode and a large reference electrode. To overcome this variability, the modern systems, such as the CellKey system (MDS Sciex, South San Francisco, Calif.) and RT-CES (ACEA Biosciences Inc., San Diego, Calif.), use an integrated circuit having a microelectrode array.

The CellKey system consists of an environmentally controlled impedance measurement system, a 96-well electrode-embedded microtiter plate, an onboard 96-well fluidics, and acquisition and analysis software. The cells are seeded in the culture wells; each well has an integrated electrode array. The system operates using a small-amplitude alternating voltage at 24 frequencies, from 1 KHz to 10 MHz. The resultant current is measured at an update rate of 2 sec. The system is thermally regulated and experiments can be conducted between 28 degrees C. and 37 degrees C. A 96-well head fluid delivery device handles fluid additions and fluid exchanges onboard.

The RT-CES system is composed of four main components: electronic microtiter plates (E-Plate), E-Plate station, electronic analyzer, and a monitoring system for data acquisition and display. The electronic analyzer sends and receives the electronic signals. The E-Plate station is placed inside a tissue culture incubator. The E-Plate station comes in three throughput varieties: a 16× station for running six 16-well E-Plates at a time, a single 96-well E-Plate station, and the Mult-E-Plate™station, which can accommodate up to six 96-well E-Plates at a time. The cells are seeded in E-Plates, which are integrated with microelectronic sensor arrays. The system operates at a low-voltage (less than 20 mV) AC signal at multiple frequencies.

Cells are dynamic objects with relatively large dimensions, typically tens of microns. RWG biosensors enable detection of ligand-induced changes within the portion of cells closest to the sensor surface, determined by the penetration depth of the evanescent wave. Furthermore, the spatial resolution of an optical biosensor is determined by the spot size (100 microns) of the incident light source. Thus, although low confluent cells can be used for assaying, a highly confluent cell layer is generally used in order to achieve optimal assay results. The sensor configuration can be viewed as a three-layer waveguide composite consisting of, for example, a substrate, waveguide thin film, and a cell layer. It has been reported that for whole-cell sensing of a ligand-induced change in effective refractive index, the detected signal is the sum of mass redistribution occurring at distinct distances away from the sensor surface, each with unequal contribution to the overall response. The reported relationship suggests that the detected signal with an RWG biosensor is sensitive primarily to the vertical mass redistribution, as a result of a change in local protein concentration. The detected signal is often referred to as a dynamic mass redistribution (DMR) signal. GPCR activation leads to a series of spatial and temporal events, including, for example, ligand binding, receptor activation, protein recruitment, receptor internalization and recycling, second messenger alternation, cytoskeletal remodeling, gene expression, and cell adhesion changes. Each cellular event has its own characteristics regarding its kinetics, duration, amplitude, and mass movement. Thus it is reasonable to assume that these cellular events may contribute differently to the overall DMR signal, depending on where they occur. Using a panel of agonists targeting a variety of GPCRs, three classes of DMR signals were identified in human epidermoid carcinoma A431 cells, which reflect the signaling pathways mediated. Since each is correlated with the activation of a class of GPCRs depending on the G protein with which the receptor is coupled, the DMR signals obtained were named G_{q}-DMR Gₛ-DMR and G,-DMR signals, respectively. Each class of DMR signals exhibits distinct kinetic and dynamic characteristics, reflecting the unique signaling integration mediated through different classes of GPCRs. The unique characteristics of the DMR signals can be used to identify the G-protein coupling mechanism activated by a GPCR stimulated by a given ligand.

Bioimpedance signals of GPCR activation—In a typical impedance-based cell assay, cells are brought into contact with a gold electrode arrayed on the bottom of culture wells. The total impedance of the sensor system is determined primarily by the ion environment surrounding the biosensor. Under application of an electrical field, the ions undergo field-directed movement and concentration gradient-driven diffusion. For whole cell sensing, the total electrical impedance has four components: the resistance of the electrolyte solution, the impedance of the cell, the impedance at the electrode/solution interface, and the impedance at the electrode/cell interface. In addition, the impedance of a cell comprises two components, the resistance and the reactance. The conductive characteristics of cellular ionic strength provide the resistive component, whereas the cell membranes, acting as imperfect capacitors, contribute a frequency-dependent reactive component. Thus, the total impedance is a function of many factors, including cell viability, cell confluency, cell numbers, cell morphology, degree of cell adhesion, ionic environment, the water content within the cells, and the detection frequency.

In the RT-CES system, a percentage of this small voltage applied is coupled into the cell interior. Such signals applied to cells are believed to be much smaller than the resting membrane potential of a typical mammalian cell and thus present minimal or no disturbance to cell function. The RT-CES system measures these changes in impedance and displays it as a parameter called the cell index.

In the CellKey system, a change in sensor system's impedance is attributed to a change in complex impedance (delta Z or dZ) of a cell layer that occurs in response to receptor stimulation. At low frequencies, the small voltage applied induces extracellular currents (iec) that pass around individual cells in the layer. However, the conduction currents through cell membrane due to ion channels may also be important at low measurement frequencies. At high frequencies, they induce transcellular currents (itc) that can penetrate the cell membrane. The ratio of the applied voltage to the measured current for each well is its impedance (Z) as described by Ohm's law.

When cells are exposed to a stimulus, such as a receptor ligand, signal transduction events are activated that lead to complex cellular events such as modulation of the actin cytoskeleton that can cause changes in cell adherence, cell shape and volume, and cell-to-cell interaction. These cellular changes individually or collectively affect the flow of extracellular and transcellular current, and therefore, affect the magnitude and characteristics of the measured impedance. At least three types of impedance signals can be mediated through the activation of three classes of GPCRs, depending on the G protein to which the receptor is coupled. The impedance profiles can be obtained using, for example, a CellKey system (www.cellkey.com). Similar profiles can be recorded using the RT-CES system. Although not limited by theory it is believed that these impedance signals are due to the different effects on the actin cytoskeleton that affect the cellular parameters measured by impedance, in response to the activation of different classes of GPCRs. The activation of G_{q} and Gᵢ GPCRs has been shown to lead to increased actin polymerization, while stimulation of Gₛ GPCRs leads to actin depolymerization.

The eukaryotic cells used in the subject assays will stably or transiently express at least one functional TAS1R or TAS2R. In some cases, the eukaryotic cell will either stably or transiently express a functional TAS1R1/TAS1R3 umami taste receptor or a functional TAS1R2/TAS1R3 sweet taste receptor or will stably or transiently express a desired functional TAS2R. In some cases, the eukaryotic cell will further express a G protein that couples with said TAS1R(s) or TAS2R, which G protein may be expressed from an endogenous gene or from a heterologous gene, such as a stably or transiently transfected gene. Examples of suitable G proteins are known in the art and are referred in the patent applications incorporated by reference herein. In certain embodiments, the G protein may comprise a Gi protein selected from Gαi, i.e., Gαi 1-1, Gαi 1-2, Gαi 1-3, Gαi 0-1, and Gαi 0-2; Gα15, a-transducin, gustducin, Gαz or a functional chimera or variant thereof that couples with the TAS1R(s) or TAS2R expressed by the eukaryotic cell. Should also include wording on G protein-independent effects.

The present assays can be effected using any eukaryotic cell that functionally expresses the particular TAS1R(s) or TAS2R, and which cell, when contacted with an activator of said TAS1R or TAS1R results in receptor activation. Examples of suitable eukaryotic cells include amphibian, yeast, insect, amphibian, worm and mammalian cells. Specific examples of suitable cells for use in the subject cell-based assays include BHK cells, U2OS cells, CHO cells, HeLa cells, and Xenopus oocytes.

In some embodiments of any of the foregoing aspects, the eukaryotic cell expresses at least one taste receptor or G protein from an endogenous gene. For example, the eukaryotic cells may express TAS1R3 from an endogenous gene; potentially suitable cell lines that express TAS1R3 (either with our without the G protein gustducin) are identified in the Examples below. The eukaryotic cell may further express one or more taste receptor or G protein from a heterologous gene. For example, a cell line that endogenously expresses one subunit of a heteromeric sweet or umami receptor may be transformed with a gene encoding the other subunit of said receptor or with a G protein that can functionally couple thereto.

The methods of the present disclosure provide cell-based assay methods that enable the identification of modulators, e.g., agonists, antagonists, inverse agonists enhancers of a TAS1R or TAS2R receptors. The TAS2R modulators are useful for altering taste perception, for example to induce, suppress or enhance bitter taste perception in a subject. The TAS1R2/TAS1R3 modulators are useful for modulating sweet taste, e.g., by enhancing the taste of another sweet tasting compound such as sucralose. The TAS1R1/TAS1R3 modulators identified are useful for modulating umami taste, e.g., by enhancing the taste of a umami compound such as monosodium glutamate.

### Taste Receptor Modulators and Comestible Compositions

In a certain aspects, the disclosure provides uses of the taste receptor modulator identified by the methods of any of the foregoing aspects or embodiments thereof to modify a taste of a comestible composition. In some embodiments thereof, the comestible composition is not a naturally occurring composition. In some embodiments thereof, modifying the taste of a comestible composition comprises enhancing a sweet taste of the comestible composition, enhancing an umami taste of a comestible composition, or inhibiting a bitter taste of a comestible composition. In some embodiments, the comestible composition is a food product, a beverage product, a pharmaceutical composition, a cosmetic composition, an oral care composition, or a detrifice.

In certain aspects, the disclosure provides comestible compositions comprising the taste receptor modulator identified by the methods of any of the foregoing aspects or embodiments thereof. In some embodiments thereof, the comestible composition is a naturally occurring composition. In some embodiments thereof, the comestible composition is not a naturally occurring composition. In some embodiments, the comestible composition is a food product, a beverage product, a pharmaceutical composition, a cosmetic composition, an oral care composition, or a detrifice.

In accordance with the methods of the present disclosure, a composition that is administered to alter taste perception in a subject will comprise an effective amount of a TAS1R or TAS2R modulator (agonist, antagonist, or enhancer). A TAS1R or TAS2R activator or modulator can comprise any substance, e.g., small molecule, peptide, protein, carbohydrate, oligosaccharide, glycoprotein, amino acid derivative, and the like. In general, compounds will be identified by screening libraries of potential taste modulatory compounds, which may be comprised of synthetic or naturally occurring compounds. The library may be random or may comprise compounds having related structures or are structures or substitutions. After lead candidates are identified, compound libraries having similar structure will be produced and screened for TAS1R or TAS2R modulatory activity according to the present disclosure. TAS1R or TAS2R modulators identified as disclosed herein can be used to prepare compositions suitable for oral use, including but not limited to food, beverages, oral washes, dentifrices, cosmetics, and pharmaceuticals. TAS1R or TAS2R modulators can also be used as additives to alter the sweet, umami or bitter taste of a compound that is of palatable but undesirable for oral use, for example compounds comprised in household cleansers, poisons, etc. Such modulators will alter bitter, sweet or umami tasting compounds contained therein.

For example, representative foods having an undesirable or bitter taste include, but are not limited to, citrus fruits such as grapefruit, orange, and lemon; vegetables such as tomato, pimento, celery, melon, carrot, potato, and asparagus; seasoning or flavoring materials such as flavor, sauces, soy sauce, and red pepper; foods originating from soybean; emulsion foods such as cream, dressing, mayonnaise, and margarine; processed marine products such as fish meat, ground fish meat, and fish eggs; nuts such as peanuts; fermented foods such as fermented soybean; meats and processed meats; pickles; noodles; soups including powdery soups; dairy products such as cheese; breads and cakes; confectioneries such as candies, chewing gum, and chocolate; and specifically prepared foods for health.

Non-limiting examples of cosmetics eliciting bitter taste (e.g., skin lotions, creams, face packs, lip sticks, foundations, shaving preparations, after-shave lotions, cleansing foams, and cleansing gels) include but are not limited to those compositions that include surfactants such as sodium alkyl sulfate and sodium monoalkyl phosphate; fragrances such as menthol, linalool, phenylethyl alcohol, ethyl propionate, geraniol, linalyl acetate and benzyl acetate; antimicrobials such as methyl paraben, propyl paraben and butyl paraben; humectants such as lactic acid and sodium lactate; alcohol-denaturating agents such as sucrose octaacetate and brucine; and astringents such as aluminum lactate.

Non-limiting examples of pharmaceuticals having a bitter taste include acetaminophen, terfenadine, guaifenesin, trimethoprim, prednisolone, ibuprofen, prednisolone sodium phosphate, methacholine, pseudoephedrine hydrochloride, phenothiazine, chlorpromazine, diphenylhydantoin, caffeine, morphine, demerol, codeine, lomotil, lidocaine, salicyclic acid, sulfonamides, chloroquine, a vitamin preparation, minerals and penicillins, neostigmine, epinephrine, albuterol, dephenhydramine, chlorpheniramine maleate, chlordiazepoxide, amitriptyline, barbiturates, diphenylhydantoin, caffeine, morphine, demerol, codeine, lomotil, lidocaine, salicyclic acid, sulfonamides, chloroquine, a vitamin preparation, minerals and penicillins.

Non-limiting examples of sweeteners which may be modulated by compounds according to the present disclosure include xylitol, sorbitol, saccharin, sucralose, sucrose, glucose, fructose, cyclamate, aspartame, monellin, rebaudioside A, rebaudioside C, stevioside, mogroside V, siamonoside I and the like, and derivatives thereof.

Non-limiting examples of umami compounds, the taste which may be modulated according to the present disclosure include L-glutamate, L-asparate, monosodium glutamate, derivatives thereof, compounds containing and the like.

These taste modulators (or taste receptor modulators) can also be used as part of prepared food, beverage, oral wash, dentifrice, cosmetic, or drug. To prepare a composition suitable for administration to a subject, a TAS1R or TAS2R modulator can be admixed with a compound, the taste of which is to be modulated in amount comprising 0.001% to 10% by weight, or from 0.01% to 8% by weight, or from 0.1% to 5% by weight, or from 0.5% to 2% by weight.

Suitable formulations include, but are not limited to, solutions, extracts, elixirs, spirits, syrups, suspensions, powders, granules, capsules, pellets, tablets, and aerosols. Optionally, a formulation can include a pharmaceutically acceptable carrier, a suspending agent, a solubilizer, a thickening agent, a stabilizer, a preservative, a flavor, a colorant, a sweetener, a perfume, or a combination thereof. TAS1R or TAS2R modulators and compositions can be presented in unit-dose or multi-dose sealed containers, such as ampules and vials.

### Uses of Taste Receptor Modulators and Related Methods

In certain aspects, the disclosure provides uses of the taste receptor modulator identified by the methods of any of the foregoing aspects or embodiments thereof to modify a taste of a comestible composition. In some embodiments thereof, the comestible composition is not a naturally occurring composition. In some embodiments thereof, the comestible composition is a naturally occurring composition. In some embodiments thereof, modifying the taste of a comestible composition comprises enhancing a sweet taste of the comestible composition, enhancing an umami taste of a comestible composition, or inhibiting a bitter taste of a comestible composition. In some embodiments, the comestible composition is a food product, a beverage product, a pharmaceutical composition, a cosmetic composition, an oral care composition, or a dentrifice.

In certain related aspects, the disclosure provides for the administration of the TAS1R or TAS2R modulators identified by the assay methods disclosed herein directly to a subject for modulation of taste perception. In some cases, a modulator is administered orally or nasally, for example, as a component of a comestible composition.

In some embodiments, an effective amount of a TAS1R or TAS2R modulator is administered to a subject. The term “effective amount” refers to an amount of a composition sufficient to modulate TAS1R or TAS2R activation or to modulate taste perception, e.g., bitter, sweet or umami taste perception. An effective amount or dose can be readily determined using in vivo assays of taste perception as are known in the art.

An effective amount can be varied so as to administer an amount of a TAS1R or TAS2R modulator that is effective to achieve the desired taste perception. The selected dosage level will depend upon a variety of factors including the activity of the TAS1R or TAS2R modulator, formulation, combination with other compositions (e.g., food, drugs, etc.), the intended use (e.g., as a food additive, dentifrice, etc.), and the physical condition and prior medical history of the subject being treated.

### EXAMPLES

### Example 1: Development of a DMR Assay for the Human Sweet Taste Receptor

Untransfected U2OS cells were cultured at 37° C. and 5% CO2 in McCoy's 5A medium (modified, GIBCO#16600) supplemented with 10% fetal bovine serum and penicillin/streptomycin. Plasmids encoding hT1R2 in pEAK10-puromycin and hT1R3 in pcDNA3.1-zeocin were linearized and transfected into U2OS cells followed by dilution and double selection in growth medium plus 0.5 μg/m1puromycin and 100 μg/mlzeocin. Individual colonies were expanded and transiently transfected with a Gα6gust25 expression construct to identify functional clones using sucralose as the agonist in a Ca2+ imaging assay. The clone with the best response and growth characteristics, R2/R3 U2OS, shows functional expression of the sweet taste receptor for greater than 30 passages. (R2/R3 U2OS cells) were seeded on Epic®384 microplates (Corning) at a density of 16,000 cells/well in 40 pt of assay media using a multidrop (Thermo Scientific). The plates were incubated in the tissue culture hood for 20 minutes to allow cells to settle evenly at the bottom of the microplate and further cultured at 37° C. in an atmosphere of 5% CO2 for 18-24 h. The next day, the cells were subjected to functional analysis using DMR. The lidded Epic® 384 microplate and polypropylene compound plate (Corning) were loaded into the carousel of the Epic® reader, the target plate was washed with DMSO matched D-PBS using the Corning Epic® Liquid Handling Accessory (LHA) and equilibrated in 25 μL of DMSO matched D-PBS for 90 minutes. After recording the baseline activity for 5 to 8 minutes, 25 μL of 2× concentrated solution of sweetener, agonist or a mixture of agonist and modulator, diluted in D-PBS, was added using the LHA and the DMR responses which measures a shift in the resonance wavelength, were monitored for an additional 30 minutes. FIG. 1A: Application of increasing concentrations of sucralose onto R2/R3 U2OS cells cause a positive DMR signal. Arrow indicates time of application. FIG. 1B: Application of increasing concentrations of sucralose onto parental U2OS cells does not cause a comparable positive DMR signal. Arrow indicates time of application. FIG. 1C: Representative sucralose dose-response analysis on R2/R3 U2OS cells and parental U2OS cells.

### Example 2: DMR Assay for the Human Sweet Taste Receptor Detects Effect of Several Sweeteners

In FIG. 2A known sweeteners and new agonists were evaluated in the DMR assay with R2/R3 U2OS cells using a dose-response analysis. In FIG. 2B the same sweeteners were then evaluated in a FLIPR-calcium mobilization assay with R2/R3 U2OS cells overexpressing Gα15.

### Example 3: Select Sweeteners and Sweet Receptor Agonists Show Bias for the FLIPR Assay

FIGS. 3A-3L describe a bias plotting analysis. Depicted sweeteners and agonists were evaluated at equimolar concentrations in the DMR assay with R2/R3 U2OS cells and in the FLIPR assay with R2/R3 U2OS cells expressing Gα15. Assay data was normalized to responses obtained with 1 mM sucralose and resulting activity values were plotted on the same graph. Relative to other sweeteners, 5819, S5227, P-4000 and Neotame are more active in the FLIPR assay.

### Example 4: G Protein Coupling Causes Bias

In FIGS. 4A-4C, S5227, neotame and P-4000 were evalutated in a FLIPR-calcium mobilization assay using R2/R3 U2OS cells overexpressing Gα16gust25, a G protein more closely related to gustducin than Gα15 and a bias plotting analysis was performed.

### Example 5: DMR Assay Detects Sweet Taste Receptor Enhancers

FIGS. 5A and 5B show the evaluation of an enhancer for the human sweet taste receptor in the DMR assay. FIG. 5A: R2/R3 U2OS cells were stimulated with increasing concentrations of sucralose in the presence and absence of 25 μM SE-2 and DMR responses were monitored on the Epic® reader. Depicted are the kinetics corresponding to the effect of SE-2 on 31.25 μM sucralose. FIG. 5B: Depiction of the full dose-responses analysis described in A. The rectangle includes data points from which the kinetics in panel A were taken.

### Example 6: Expression of a Sweet Receptor Subunit and Native G Protein in Different Cell Lines

This example, shown in FIGS. 6A and 6B, identifies human cell lines expressing TAS1R3 or the G protein gustducin. These cell lines are potentially suitable for use in a label-free cell-based assay for detection of sweet or umami modulator compounds (optionally after transformation with a gene encoding TAS1R2 or TAS1R1, respectively). mRNA was isolated from cultured cell lines and RT-PCR was conducted with specific primers designed to amplify TAS1R3 and gustducin mRNA and genomic DNA, followed by agarose gel electrophoresis to detect PCR products. A GAPDH product was also amplified in separate reactions to confirm mRNA integrity. In some cases, RT-PCR results are shown in FIG. 6A-6B. A subset of cell lines from each of three origins (pancreas, colon, and colorectal tissues) expressed TAS1R3 or gustducin. Specifically, expression of TAS1R3 (with or without gustducin) was detected in the cell lines Panc-1, ASPC-1, BxPc-3, HuTu-80, HCT-8, HT-29, SW48, COLO205, COLO320-DM, HCT-15, NCI-H716, CCD841 CoN, CCD-112 CoN and CCD-33Co. Expression of both TAS1R3 and gustducin was detected in the cell lines Panc-1, HuTu-80, Sw48,CCD-112 CoN, and NCI-H716. The cell line NCI-H716 exhibited particularly strong expression of both TAS1R3 and gustducin.

### Example 7

Cell lines Panc-1, ASPC-1, BxPc-3, HuTu-80, HCT-8, HT-29, SW48, COLO205, COLO320-DM, HCT-15, NCI-H716, CCD841 CoN, CCD-112 CoN and CCD-33Co and other cell lines identified as expressing TAS1R3 are assayed using the EPIC system (as described in Example 1) to detect GPCR activation in response to sweet ligands or umami ligands. The cells (as well as other cell lines) optionally are further screened to identify cell lines that express a TAS1R2/TAS1R3 or TAS1R1/TAS1R3 receptor and/or gustducin (or another G protein such as a Gi protein) from endogenous genes. Alternatively, cells expressing a subset of TAS1R genes or G proteins are transformed to produce a cell line that transiently or stably expresses a TAS1R2/TAS1R3 or TAS1R1/TAS1R3 receptor and a G protein, resulting in a cell line that expresses a functional sweet or umami taste receptor and responds to sweet or umami ligands. Optionally, G proteins expressed by the cell lines are identified. Cell lines are optionally further transformed with a G protein construct that results in transient or stable expression of one or more G proteins by the cells.

The EPIC system is used to measure GPCR activation in the cell lines for a variety of compounds, including known agonists of the sweet or umami taste receptor expressed by the cells, optionally in the presence of known sweet or umami enhancers. The EPIC system results indicate which of the G protein pathways (Gi, Gq, Gs, and/or G12/G13) are activated. Cell lines are additionally tested for their responses to known “false positive” sweet or umami agonists or enhancers to identify any that may distinguish between true and false positive agonists or enhancers, e.g., by having a different magnitude of GPCR activation or by relative levels of activation of the different G protein pathways. Cell lines that respond to sweet or umami agonists and enhancers (for example, to distinguish between true and false positives) are then utilized in screening assays for identification of novel sweet or umami modulators. These screening assays generally comprise contacting the cells with one or more candidate modulators, optionally in the presence of a known sweet or umami activator or inhibitor, and detecting a putative modulator by its effect on GPCR activity, e.g., increased GPCR activity indicates a putative agonist or enhancer; and decreased GPCR activity indicates a putative antagonist or blocker. Putative modulators are further tested in secondary assays that may include taste tests.

Similar experiments are conducted to identify cells that express one or more bitter receptors of the TAS2R family (and optionally one or more G proteins) from endogenous genes. Cells are optionally transformed to cause stable or transient expression of one or more TAS2R and/or G protein genes. Cell lines are then tested using the EPIC system for their responses to known modulators of the TAS2R receptor, and cells that respond to known ligands and blockers are utilized in screening assays for identification of putative modulatory compounds, which are further tested in secondary assays that may include taste tests.

### Amino Acid Sequences

Listed below are the amino acid sequences for various taste receptor proteins set forth herein. The sequences are also set forth in the sequence listing filed herewith and hereby incorporated by reference into the present disclosure.

The sequence for the protein identified as TAS1R1 (or, alternatively, as hT1R1) is as follows and is also identified herein as SEQ ID NO: 1. MLLCTARLVGLQLLISCCWAFAC HSTESSPDFTLPGDYLLAGLFPLHSGCLQVRHRPEVTLCDRSCSFNEHGYHLFQAMR LGVEEINNSTALLPNITLGYQLYDVCSDSANVYATLRVLSLPGQHHIELQGDLLHYSP TVLAVIGPDSTNRAATTAALLSPFLVPMISYAASSETLSVKRQYPSFLRTIPNDKYQVE TMVLLLQKFGWTWISLVGSSDDYGQLGVQALENQATGQGICIAFKDIMPFSAQVGD ERMQCLMRHLAQAGATVVVVFSSRQLARVFFESVVLTNLTGKVWVASEAWALSRH ITGVPGIQRIGMVLGVAIQKRAVPGLKAFEEAYARADKKAPRPCHKGSWCSSNQLCR ECQAFMAHTMPKLKAFSMSSAYNAYRAVYAVAHGLHQLLGCASGACSRGRVYPW QLLEQIHKVHFLLHKDTVAFNDNRDPLSSYNIIAWDWNGPKWTFTVLGSSTWSPVQ LNINETKIQWHGKDNQVPKSVCSSDCLEGHQRVVTGFHHCCFECVPCGAGTFLNKS DLYRCQPCGKEEWAPEGSQTCFPRTVVFLALREHTSWVLLAANTLLLLLLLGTAGLF AWHLDTPVVRSAGGRLCFLMLGSLAAGSGSLYGFFGEPTRPACLLRQALFALGFTIF LSCLTVRSFQLIIIFKFSTKVPTFYHAWVQNHGAGLFVMISSAAQLLICLTWLVVWTP LPAREYQRFPHLVMLECTETNSLGFILAFLYNGLLSISAFACSYLGKDLPENYNEAKC VTFSLLFNFVSWIAFFTTASVYDGKYLPAANMMAGLSSLSSGFGGYFLPKCYVILCRP DLNSTEHFQASIQDYTRRCGST.

The sequence for the protein identified as TAS1R2 (or, alternatively, as hT1R2) is as follows and is also identified herein as SEQ ID NO: 2. MGPRAKTICSLFFLLWVLAEPAE NSDFYLPGDYLLGGLFSLHANMKGIVHLNFLQVPMCKEYEVKVIGYNLMQAMRFA VEEINNDSSLLPGVLLGYEIVDVCYISNNVQPVLYFLAHEDNLLPIQEDYSNYISRVVA VIGPDNSESVMTVANFLSLFLLPQITYSAISDELRDKVRFPALLRTTPSADHHVEAMV QLMLHFRWNWIIVLVSSDTYGRDNGQLLGERVARRDICIAFQETLPTLQPNQNMTSE ERQRLVTIVDKLQQSTARVVVVFSPDLTLYHFFNEVLRQNFTGAVWIASESWAIDPV LHNLTELGHLGTFLGITIQSVPIPGFSEFREWGPQAGPPPLSRTSQSYTCNQECDNCLN ATLSFNTILRLSGERVVYSVYSAVYAVAHALHSLLGCDKSTCTKRVVYPWQLLEEIW KVNFTLLDHQIFFDPQGDVALHLEIVQWQWDRSQNPFQSVASYYPLQRQLKNIQDIS WHTVNNTIPMSMCSKRCQSGQKKKPVGIHVCCFECIDCLPGTFLNHTEDEYECQACP NNEWSYQSETSCFKRQLVFLEWHEAPTIAVALLAALGFLSTLAILVIFWRHFQTPIVR SAGGPMCFLMLTLLLVAYMVVPVYVGPPKVSTCLCRQALFPLCFTICISCIAVRSFQI VCAFKMASRFPRAYSYWVRYQGPYVSMAFITVLKMVIVVIGMLATGLSPTTRTDPD DPKITIVSCNPNYRNSLLFNTSLDLLLSVVGFSFAYMGKELPTNYNEAKFITLSMTFYF TSSVSLCTFMSAYSGVLVTIVDLLVTVLNLLAISLGYFGPKCYMILFYPERNTPAYFN SMIQGYTMRRD.

The sequence for the protein identified as TAS1R3 (or, alternatively, as hT1R3) is as follows and is also identified herein as SEQ ID NO: 3. MLGPAVLGLSLWALLHPGT GAPLCLSQQLRMKGDYVLGGLFPLGEAEEAGLRSRTRPSSPVCTRFSSNGLLWALA MKMAVEEINNKSDLLPGLRLGYDLFDTCSEPVVAMKPSLMFLAKAGSRDIAAYCNY TQYQPRVLAVIGPHSSELAMVTGKFFSFFLMPQVSYGASMELLSARETFPSFFRTVPS DRVQLTAAAELLQEFGWNWVAALGSDDEYGRQGLSIFSALAAARGICIAHEGLVPLP RADDSRLGKVQDVLHQVNQSSVQVVLLFASVHAAHALFNYSISSRLSPKVWVASEA WLTSDLVMGLPGMAQMGTVLGFLQRGAQLHEFPQYVKTHLALATDPAFCSALGER EQGLEEDVVGQRCPQCDCITLQNVSAGLNHHQTFSVYAAVYSVAQALHNTLQCNAS GCPAQDPVKPWQLLENMYNLTFHVGGLPLRFDSSGNVDMEYDLKLWVWQGSVPR LHDVGRFNGSLRTERLKIRWHTSDNQKPVSRCSRQCQEGQVRRVKGFHSCCYDCVD CEAGSYRQNPDDIACTFCGQDEWSPERSTRCFRRRSRFLAWGEPAVLLLLLLLSLAL GLVLAALGLFVHHRDSPLVQASGGPLACFGLVCLGLVCLSVLLFPGQPSPARCLAQQ PLSHLPLTGCLSTLFLQAAEIFVESELPLSWADRLSGCLRGPWAWLVVLLAMLVEVA LCTWYLVAFPPEVVTDWHMLPTEALVHCRTRSWVSFGLAHATNATLAFLCFLGTFL VRSQPGRYNRARGLTFAMLAYFITWVSFVPLLANVQVVLRPAVQMGALLLCVLGIL AAFHLPRCYLLMRQPGLNTPEFFLGGGPGDAQGQNDGNTGNQGKHE.

The sequence for the protein identified as TAS2R1 (or, alternatively, as hT2R1) is as follows and is also identified herein as SEQ ID NO: 4. MLESHLIIYFLLAVIQFLLGIF TNGIIVVVNGIDLIKHRKMAPLDLLLSCLAVSRIFLQLFIFYVNVIVIFFIEFIMCSANCA ILLFINELELWLATWLGVFYCAKVASVRHPLFIWLKMRISKLVPWMILGSLLYVSMIC VFHSKYAGFMVPYFLRKFFSQNATIQKEDTLAIQIFSFVAEFSVPLLIFLFAVLLLIFSL GRHTRQMRNTVAGSRVPGRGAPISALLSILSFLILYFSHCMIKVFLSSLKFHIRRFIFLF FILVIGIYPSGHSLILILGNPKLKQNAKKFLLHSKCCQ.

The sequence for the protein identified as TAS2R3 (or, alternatively, as hT2R3) is as follows and is also identified herein as SEQ ID NO: 5. MMGLTEGVFLILSGTQFTLGIL VNCFIELVNGSSWFKTKRMSLSDFIITTLALLRIILLCIILTDSFLIEFSPNTHDSGIIMQII DVSWTFTNHLSIWLATCLGVLYCLKIASFSHPTFLWLKWRVSRVMVWMLLGALLLS CGSTASLINEFKLYSVFRGIEATRNVTEHFRKKRSEYYLIHVLGTLWYLPPLIVSLASY SLLIFSLGRHTRQMLQNGTSSRDPTTEAHKRAIRIILSFFFLFLLYFLAFLIASFGNFLPK TKMAKMIGEVMTMFYPAGHSFILILGNSKLKQTFVVMLRCESGHLKPGSKGPIFS.

The sequence for the protein identified as TAS2R4 (or, alternatively, as hT2R4) is as follows and is also identified herein as SEQ ID NO: 6. MLRLFYFSAIIASVILNFVG IIMNLFITVVNCKTWVKSHRISSSDRILFSLGITRFLMLGLFLVNTIYFVSSNTERSVYL SAFFVLCFMFLDSSSVWFVTLLNILYCVKITNFQHSVFLLLKRNISPKIPRLLLACVLIS AFTTCLYITLSQASPFPELVTTRNNTSFNISEGILSLVVSLVLSSSLQFIINVTSASLLIHS LRRHIQKMQKNATGFWNPQTEAHVGAMKLMVYFLILYIPYSVATLVQYLPFYAGM DMGTKSICLIFATLYSPGHSVLIIITHPKLKTTAKKILCFKK.

The sequence for the protein identified as TAS2R5 (or, alternatively, as hT2R5) is as follows and is also identified herein as SEQ ID NO: 7. MLSAGLGLLMLVAVVEFLIGL IGNGILVVWSFREWIRKFNWSSYNLIILGLAGCRFLLQWLIILDLSLFPLFQSSRWLRY LSIFWVLVSQASLWFATFLSVFYCKKITTFDRPAYLWLKQRAYNLSLWCLLGYFIINL LLTVQIGLTFYHPPQGNSSIRYPFESWQYLYAFQLNSGSYLPLVVFLVSSGMLIVSLYT HHKKMKVHSAGRRDVRAKAHITALKSLGCFLLLHLVYIMASPFSITSKTYPPDLTSV FIWETLMAAYPSLHSLILIMGIPRVKQTCQKILWKTVCARRCWGP.

The sequence for the protein identified as TAS2R7 (or, alternatively, as hT2R7) is as follows and is also identified herein as SEQ ID NO: 8. MADKVQTTLLFLAVGEFSV GILGNAFIGLVNCMDWVKKRKIASIDLILTSLAISRICLLCVILLDCFILVLYPDVYATG KEMRIIDFFWTLTNHLSIWFATCLSIYYFFKIGNFFHPLFLWMKWRIDRVISWILLGCV VLSVFISLPATENLNADFRFCVKAKRKTNLTWSCRVNKTQHASTKLFLNLATLLPFC VCLMSFFLLILSLRRHIRRMQLSATGCRDPSTEAHVRALKAVISFLLLFIAYYLSFLIAT SSYFMPETELAVIFGESIALIYPSSHSFILILGNNKLRHASLKVIWKVMSILKGRKFQQH KQI.

The sequence for the protein identified as TAS2R8 (or, alternatively, as hT2R8) is as follows and is also identified herein as SEQ ID NO: 9. MFSPADNIFIILITGEFILGILG NGYIALVNWIDWIKKKKISTVDYILTNLVIARICLISVMVVNGIVIVLNPDVYTKNKQ QIVIFTFWTFANYLNMWITTCLNVFYFLKIASSSHPLFLWLKWKIDMVVHWILLGCF AISLLVSLIAAIVLSCDYRFHAIAKHKRNITEMFHVSKIPYFEPLTLFNLFAIVPFIVSLIS FFLLVRSLWRHTKQIKLYATGSRDPSTEVHVRAIKTMTSFIFFFFLYYISSILMTFSYL MTKYKLAVEFGEIAAILYPLGHSLILIVLNNKLRQTFVRMLTCRKIACMI.

The sequence for the protein identified as TAS2R9 (or, alternatively, as hT2R9) is as follows and is also identified herein as SEQ ID NO: 10. MPSAIEAIYIILIAGELTIGIWGN GFIVLVNCIDWLKRRDISLIDIILISLAISRICLLCVISLDGFFMLLFPGTYGNSVLVSIVN VVWTFANNSSLWFTSCLSIFYLLKIANISHPFFFWLKLKINKVMLAILLGSFLISLIISVP KNDDMWYHLFKVSHEENITWKFKVSKIPGTFKQLTLNLGAMVPFILCLISFFLLLFSL VRHTKQIRLHATGFRDPSTEAHMRAIKAVIIFLLLLIVYYPVFLVMTSSALIPQGKLVL MIGDIVTVIFPSSHSFILIMGNSKLREAFLKMLRFVKCFLRRRKPFVP.

The sequence for the protein identified as TAS2R10 (or, alternatively, as hT2R10) is as follows and is also identified herein as SEQ ID NO: 11. MLRVVEGIFIFVVVSESVFGV LGNGFIGLVNCIDCAKNKLSTIGFILTGLAISRIFLIWIIITDGFIQIFSPNIYASGNLIEYIS YFWVIGNQSSMWFATSLSIFYFLKIANFSNYIFLWLKSRTNMVLPFMIVFLLISSLLNF AYIAKILNDYKMKNDTVWDLNMYKSEYFIKQILLNLGVIFFFTLSLITCIFLIISLWRH NRQMQSNVTGLRDSNTEAHVKAMKVLISFIILFILYFIGMAIEISCFTVRENKLLLMFG MTTTAIYPWGHSFILILGNSKLKQASLRVLQQLKCCEKRKNLRVT.

The sequence for the protein identified as TAS2R13 (or, alternatively, as hT2R13) is as follows and is also identified herein as SEQ ID NO: 12. MESALPSIFTLVIIAEFIIGNLS NGFIVLINCIDWVSKRELSSVDKLLIILAISRIGLIWEILVSWFLALHYLAIFVSGTGLRI MIFSWIVSNHFNLWLATIFSIFYLLKIASFSSPAFLYLKWRVNKVILMILLGTLVFLFLN LIQINMHIKDWLDRYERNTTWNFSMSDFETFSVSVKFTMTMFSLTPFTVAFISFLLLIF SLQKHLQKMQLNYKGHRDPRTKVHTNALKIVISFLLFYASFFLCVLISWISELYQSTVI YMLCETIGVFSPSSHSFLLILGNAKLRQAFLLVAAKVWAKR.

The sequence for the protein identified as TAS2R14 (or, alternatively, as hT2R14) is as follows and is also identified herein as SEQ ID NO: 13. MGGVIKSIFTFVLIVEFIIGNL GNSFIALVNCIDWVKGRKISSVDRILTALAISRISLVWLIFGSWCVSVFFPALFATEKM FRMLTNIWTVINHFSVWLATGLGTFYFLKIANFSNSIFLYLKWRVKKVVLVLLLVTS VFLFLNIALINIHINASINGYRRNKTCSSDSSNFTRFSSLIVLTSTVFIFIPFTLSLAMFLL LIFSMWKHRKKMQHTVKISGDASTKAHRGVKSVITFFLLYAIFSLSFFISVWTSERLE ENLIILSQVMGMAYPSCHSCVLILGNKKLRQASLSVLLWLRYMFKDGEPSGHKEFRE SS.

The sequence for the protein identified as TAS2R16 (or, alternatively, as hT2R16) is as follows and is also identified herein as SEQ ID NO: 14. MIPIQLTVFFMIIYVLESLTIIV QSSLIVAVLGREWLQVRRLMPVDMILISLGISRFCLQWASMLNNFCSYFNLNYVLCN LTITWEFFNILTFWLNSLLTVFYCIKVSSFTHHIFLWLRWRILRLFPWILLGSLMITCVT IIPSAIGNYIQIQLLTMEHLPRNSTVTDKLENFHQYQFQAHTVALVIPFILFLASTIFLM ASLTKQIQHHSTGHCNPSMKAHFTALRSLAVLFIVFTSYFLTILITIIGTLFDKRCWLW VWEAFVYAFILMHSTSLMLSSPTLKRILKGKC.

The sequence for the protein identified as TAS2R30 (or, alternatively, as hT2R44) is as follows and is also identified herein as SEQ ID NO: 15. MITFLPIIFSILIVVIFVIGNFAN GFIALVNSIEWVKRQKISFVDQILTALAVSRVGLLWVLLLHWYATQLNPAFYSVEVR ITAYNVWAVTNHFSSWLATSLSMFYLLRIANFSNLIFLRIKRRVKSVVLVILLGPLLFL VCHLFVINMDETVWTKEYEGNVTWKIKLRSAMYHSNMTLTMLANFVPLTLTLISFL LLICSLCKHLKKMQLHGKGSQDPSTKVHIKALQTVTSFLLLCAIYFLSMIISVCNLGRL EKQPVFMFCQAIIFSYPSTHPFILILGNKKLKQIFLSVLRHVRYWVKDRSLRLHRFTRG ALCVF.

The sequence for the protein identified as TAS2R38 (or, alternatively, as hT2R51) is as follows and is also identified herein as SEQ ID NO: 16. MLTLTRIRTVSYEVRSTFLFIS VLEFAVGFLTNAFVFLVNFWDVVKRQPLSNSDCVLLCLSISRLFLHGLLFLSAIQLTH FQKLSEPLNHSYQAIIMLWMIANQANLWLAACLSLLYCSKLIRFSHTFLICLASWVSR KISQMLLGIILCSCICTVLCVWCFFSRPHFTVTTVLFMNNNTRLNWQIKDLNLFYSFLF CYLWSVPPFLLFLVSSGMLTVSLGRHMRTMKVYTRNSRDPSLEAHIKALKSLVSFFC FFVISSCAAFISVPLLILWRDKIGVMVCVGIMAACPSGHAAVLISGNAKLRRAVMTIL LWAQSSLKVRADHKADSRTLC.

The sequence for the protein identified as TAS2R39 (or, alternatively, as hT2R54) is as follows and is also identified herein as SEQ ID NO: 17. MTKLCDPAESELSPFLITLILA VLLAEYLIGIIANGFIMAIHAAEWVQNKAVSTSGRILVFLSVSRIALQSLMMLEITISST SLSFYSEDAVYYAFKISFIFLNFCSLWFAAWLSFFYFVKIANFSYPLFLKLRWRITGLIP WLLWLSVFISFSHSMFCINICTVYCNNSFPIHSSNSTKKTYLSEINVVGLAFFFNLGIVT PLIMFILTATLLILSLKRHTLHMGSNATGSNDPSMEAHMGAIKAISYFLILYIFNAVAL FIYLSNMFDINSLWNNLCQIIMAAYPASHSILLIQDNPGLRRAWKRLQLRLHLYPKEW TL.

The sequence for the protein identified as TAS2R40 (or, alternatively, as hT2R55) is as follows and is also identified herein as SEQ ID NO: 18. MATVNTDATDKDISKFKVTF TLVVSGIECITGILGSGFITAIYGAEWARGKTLPTGDRIMLMLSFSRLLLQIWMMLENI FSLLFRIVYNQNSVYILFKVITVFLNHSNLWFAAWLKVFYCLRIANFNHPLFFLMKRK IIVLMPWLLRLSVLVSLSFSFPLSRDVFNVYVNSSIPIPSSNSTEKKYFYETNMVNLVFF YNMGIFVPLIMFILAATLLILSLKRHTLHMGSNATGSRDPSMKAHIGAIKATSYFLILY IFNAIALFLSTSNIFDTYSSWNILCKIIMAAYPAGHSVQLILGNPGLRRAWKRFQHQVP LYLKGQTL.

The sequence for the protein identified as TAS2R43 (or, alternatively, as hT2R61) is as follows and is also identified herein as SEQ ID NO: 19. MITFLPIIFSSLVVVTFVIGNF ANGFIALVNSIEWFKRQKISFADQILTALAVSRVGLLWVLLLNWYSTVLNPAFNSVE VRTTAYNIWAVINHFSNWLATTLSIFYLLKIANFSNFIFLHLKRRVKSVILVMLLGPLL FLACHLFVINMNEIVRTKEFEGNMTWKIKLKSAMYFSNMTVTMVANLVPFTLTLLSF MLLICSLCKHLKKMQLHGKGSQDPSTKVHIKALQTVISFLLLCAIYFLSIMISVWSFGS LENKPVFMFCKAIRFSYPSIHPFILIWGNKKLKQTFLSVFWQMRYWVKGEKTSSP.

The sequence for the protein identified as TAS2R20 (or, alternatively, as hT2R63) is as follows and is also identified herein as SEQ ID NO: 20. MMSFLHIVFSILVVVAFILGN FANGFIALINFIAWVKRQKISSADQIIAALAVSRVGLLWVILLHWYSTVLNPTSSNLK VIIFISNAWAVTNHFSIWLATSLSIFYLLKIVNFSRLIFHHLKRKAKSVVLVIVLGSLFFL VCHLVMKHTYINVWTEECEGNVTWKIKLRNAMHLSNLTVAMLANLIPFTLTLISFLL LIYSLCKHLKKMQLHGKGSQDPSTKIHIKALQTVTSFLILLAIYFLCLIISFWNFKMRP KEIVLMLCQAFGIIYPSFHSFILIWGNKTLKQTFLSVLWQVTCWAKGQNQSTP.

The sequence for the protein identified as TAS2R31 (or, alternatively, as hT2R64) is as follows and is also identified herein as SEQ ID NO: 21. MTTFIPIIFSSVVVVLFVIGNF ANGFIALVNSIEWVKRQKISFADQILTALAVSRVGLLWVLLLNWYSTVFNPAFYSVE VRTTAYNVWAVTGHFSNWLATSLSIFYLLKIANFSNLIFLHLKRRVKSVILVMLLGPL LFLACQLFVINMKEIVRTKEYEGNMTWKIKLRSAVYLSDATVTTLGNLVPFTLTLLC FLLLICSLCKHLKKMQLHGKGSQDPSTKVHIKVLQTVIFFLLLCAIYFLSIMISVWSFG SLENKPVFMFCKAIRFSYPSIHPFILIWGNKKLKQTFLSVLRQVRYWVKGEKPSSP.

The sequence for the protein identified as TAS2R19 (or, alternatively, as hT2R65) is as follows and is also identified herein as SEQ ID NO: 22. MMCFLLIISSILVVFAFVLGNV ANGFIALVNIIDWVNTRKISSAEQILTALVVSRIGLLWVMLFLWYATVFNSALYGLEV RIVASNAWAVTNHFSMWLAASLSIFCLLKIANFSNLISLHLKKRIKSVVLVILLGPLVF LICNLAVITMDERVWTKEYEGNVTWKIKLRNAIHLSSLTVTTLANLIPFTLSLICFLLLI CSLCKHLKKMRLHSKGSQDPSTKVHIKALQTVTSFLMLFAIYFLCIITSTWNLRTQQS KLVLLLCQTVAIMYPSFHSFILIMGSRKLKQTFLSVLWQMTR.

The sequence for the protein identified as TAS2R50 (or, alternatively, as hT2R67) is as follows and is also identified herein as SEQ ID NO: 23. MITFLYIFFSILIMVLFVLGNF ANGFIALVNFIDWVKRKKISSADQILTALAVSRIGLLWALLLNWYLTVLNPAFYSVEL RITSYNAWVVTNHFSMWLAANLSIFYLLKIANFSNLLFLHLKRRVRSVILVILLGTLIF LVCHLLVANMDESMWAEEYEGNMTGKMKLRNTVHLSYLTVTTLWSFIPFTLSLISF LMLICSLCKHLKKMQLHGEGSQDLSTKVHIKALQTLISFLLLCAIFFLFLIVSVWSPRR LRNDPVVMVSKAVGNIYLAFDSFILIWRTKKLKHTFLLILCQIRC.

The sequence for the protein identified as TAS2R41 (or, alternatively, as hT2R71) is as follows and is also identified herein as SEQ ID NO: 24. MQAALTAHFFVLLFSLLSLLGI AANGFIVLVLGREWLRYGRLLPLDMILISLGASRFCLQLVGTVHNFYYSAQKVEYSG GLGRQFFHLHWHFLNSATFWFCSWLSVLFCVKIANITHSTFLWLKWRFPGWVPWLL LGSVLISFIITLLFFWVNYPVYQEFLIRKFSGNMTYKWNTRIETYYFPSLKLVIWSIPFS VFLVSIMLLINSLRRHTQRMQHNGHSLQDPSTQAHTRALKSLISFLILYALSFLSLIIDA AKFISMQNDFYWPWQIAVYLCISVHPFILIFSNLKLRSVFSQLLLLARGFWVA.

The sequence for the protein identified as TAS2R46 (or, alternatively, as hT2R75) is as follows and is also identified herein as SEQ ID NO: 25. MITFLPIIFSILIVVTFVIGNFA NGFIALVNSIEWFKRQKISFADQILTALAVSRVGLLWVLVLNWYATELNPAFNSIEVR ITAYNVWAVINHFSNWLATSLSIFYLLKIANFSNLIFLHLKRRVKSVVLVILLGPLLFL VCHLFVINMNQIIWTKEYEGNMTWKIKLRSAMYLSNTTVTILANLVPFTLTLISFLLLI CSLCKHLKKMQLHGKGSQDPSMKVHIKALQTVTSFLLLCAIYFLSIIMSVWSFESLEN KPVFMFCEAIAFSYPSTHPFILIWGNKKLKQTFLSVLWHVRYWVKGEKPSSS.

1. A cell-based method of detecting a taste receptor modulating compound, the method comprising:
(a) providing a eukaryotic cell that expresses a taste receptor and, optionally, a G protein that functionally couples to the taste receptor, wherein the taste receptor comprises a G-coupled protein receptor (GPCR) selected from the group consisting of TAS1R proteins, TAS2R proteins, and combinations thereof;
(b) contacting the eukaryotic cell with a test compound that potentially modulates the activity of the taste receptor;
(c) measuring a functional effect of the test compound on the eukaryotic cell using an assay method that comprises using a biosensor to detect or measure a change in cell mass distribution, impedance, or a combination thereof; and
(d) identifying the test compound as a taste receptor modulator based on the functional effect.

2. A cell-based method of detecting a taste receptor modulating compound, the method comprising:
(a) providing a eukaryotic cell that expresses a taste receptor and, optionally, a G protein that functionally couples to the taste receptor, wherein the taste receptor comprises a G-coupled protein receptor (GPCR) selected from the group consisting of TAS1R proteins, TAS2R proteins, and combinations thereof;
(b) contacting the eukaryotic cell with a test compound that potentially modulates the activity of the taste receptor;
(c) measuring a functional effect of the test compound on the eukaryotic cell using an assay method that comprises detecting or measuring a change in GPCR activity, wherein the change in GPCR activity is not indicated through the use of a label or a dye; and
(d) identifying the test compound as a taste receptor modulator based on the functional effect.

3. A cell-based method of detecting a taste receptor modulator compound, the method comprising:
(a) providing a eukaryotic cell that expresses a taste receptor and, optionally, a G protein that functionally couples to the taste receptor, wherein the taste receptor comprises a G-coupled protein receptor (GPCR) selected from the group consisting of TAS1R proteins, TAS2R proteins, and combinations thereof;
(b) contacting the eukaryotic cell with an identified taste receptor modulator and a test compound that potentially modulates an effect of the identified taste receptor modulator on activity of the taste receptor;
(c) measuring a functional effect of the test compound on the eukaryotic cell using an assay method that comprises using a biosensor to detect or measure a change in cell mass distribution, impedance, or a combination thereof; and
(d) identifying the test compound as a taste receptor modulator based on the functional effect.

4. A cell-based method of detecting a taste receptor modulator compound, the method comprising:
(a) providing a eukaryotic cell that expresses a taste receptor and, optionally, a G protein that functionally couples to the taste receptor, wherein the taste receptor comprises a G-coupled protein receptor (GPCR) selected from the group consisting of TAS1R proteins, TAS2R proteins, and combinations thereof;
(b) contacting the eukaryotic cell with an identified taste receptor modulator and a test compound that potentially modulates an effect of the identified taste receptor modulator on activity of the taste receptor;
(c) measuring a functional effect of the test compound on the eukaryotic cell using an assay method comprising detecting or measuring a change in GPCR activity, wherein the change in GPCR activity is not indicated through the use of a label or a dye; and
(d) identifying the test compound as a taste receptor modulator based on the functional effect.

5. The method of any one of claims 1 to 4, wherein said taste receptor is an umami taste receptor.

6. The method of any one of any one of claims 1 to 5, wherein the taste receptor comprises a TAS1R1 protein, a TAS1R3 protein or a combination thereof

7. The method of claim 5 or 6, comprising identifying the test compound as a compound that putatively activates umami taste sensation.

8. The method of any one of claims 3 to 7, wherein the identified taste modulator compound is a compound that activated umami taste sensation.

9. The method of claim 8, wherein the identified taste modulator compound is monosodium glutamate, L-aspartate, L-glutamate, or any combination thereof.

10. The method of any one of claims 3 to 9, comprising identifying the test compound as a compound that putatively enhances umami taste of the identified taste modulator compound based on the functional effect.

11. The method of any one of claims 3 to 9, comprising identifying the test compound as a compound that putatively reduces umami taste of the identified taste modulator compound based on the functional effect.

12. The method of any one of claims 1 to 4, wherein said taste receptor is a sweet taste receptor.

13. The method of any one of any one of claims 1 to 4, wherein the taste receptor comprises a TAS1R2 protein, a TAS1R3 protein, or a combination thereof.

14. The method of claim 12 or 13, comprising identifying the test compound as a compound that putatively activates sweet taste sensation.

15. The method of any one of claims 3, 4, and 12-14, wherein the identified taste modulator compound is a compound that activates sweet taste sensation.

16. The method of claim 15, wherein the identified taste modulator compound is a natural sweetener, artificial sweetener, saccharin, glucose, sucrose, sucralose, sorbitol, xylose, dextran, aspartame, monellin, cyclamate, fructose, treholose, D-tryptophan, xorbitol, xylitol, L-tryptophan, rebaudioside A, rebaudioside C, stevioside, or any combination thereof.

17. The method of any one of claims 3, 4, and 12-16, comprising identifying the test compound as a compound that putatively enhances sweet taste of the identified taste modulator compound based on the functional effect.

18. The method of any one of claims 3, 4, and 12-16, comprising identifying the test compound as a compound that putatively reduces sweet taste of the identified taste modulator compound based on the functional effect.

19. The method of any one of claims 1 to 4, wherein said taste receptor is a bitter taste receptor.

20. The method of any one of any one of claims 1 to 4, wherein the taste receptor comprises a TAS2R protein, such as a TAS2R1 protein, a TAS2R3 protein, a TAS2R4 protein, a TAS2R5 protein, a TAS2R7 protein, a TAS2R8 protein, a TAS2R9 protein, a TAS2R10 protein, a TAS2R13 protein, a TAS2R14 protein, a TAS2R16 protein, a TAS2R30 protein, a TAS2R38 protein, a TAS2R39 protein, a TAS2R40 protein, a TAS2R43 protein, a TAS2R20 protein, a TAS2R31 protein, a TAS2R19 protein, a TAS2R50 protein, a TAS2R41 protein, a TAS2R46 protein, or any combinations thereof.

21. The method of claim 19 or 20, comprising identifying the test compound as a compound that putatively inhibits bitter taste sensation.

22. The method of any one of claims 3, 4, and 19-21, wherein the identified taste modulator compound is a compound that inhibits bitter taste sensation.

23. The method of claim 22, wherein the identified taste modulator compound is quinine, denatonium, lidocaine, caffeine, andrographolide, dilthiazem, chloroquine, ofloxacin, oxyphenonium, aristolocihc acid, ranitidine, cycloheximide, strychnine, salicin, phenylthiocarbamide, or mixtures thereof.

24. The method of any one of claims 3, 4, and 19-23, comprising identifying the test compound as a compound that putatively enhances bitter taste of the identified taste modulator compound based on the functional effect.

25. The method of any one of claims 3, 4, and 19-23, comprising identifying the test compound as a compound that putatively reduces bitter taste of the identified taste modulator compound based on the functional effect.

26. The method of any one of claims 1, 3, and 5 to 25, wherein using a biosensor to detect or measure a change in cell mass distribution, impedance, or a combination thereof comprise using an optical waveguide grating or impedance-based grating to quantify changes in cell mass distribution or impedance at a focal point near a substratum to which the eukaryotic cell is adhered.

27. The method of any one of claims 2, 4, and 5 to 25, wherein using an assay method comprising detecting or measuring a change in GPCR activity comprises detecting or measuring activation of the G protein, wherein the G protein is a member of a class selected from the group consisting of a Gi protein, a Gq protein, a Gs protein, and a G12/G13 protein.

28. The method of any one of claims 1 to 27, wherein the G protein is a Gi protein.

29. The method of any one of claims 1 to 28, wherein the G protein is a Gαi protein, a Gαi 1-1 protein, a Gαi 1-2 protein, a Gαi 1-3 protein, a Gαi 0-1 protein, a Gαi 0-2 protein, an a-transducin protein, a gustducin protein, a Gaz protein, or a functional chimera or variant thereof.

30. The method of any one of claims 27 to 29, wherein detecting or measuring activation of the G protein comprises identifying a G protein pathway that is activated, which is selected from the group consisting of a Gi protein pathway, a Gq protein pathway, a Gs protein pathway, and a G12/G13 protein pathway.

31. The method of any one of claims 1 to 31, wherein the eukaryotic cell stably or transiently expresses the taste receptor.

32. The method of any one of claims 1 to 31, wherein the eukaryotic cell comprises an insect cell, an amphibian cell, a yeast cell, a worm cell, or a mammalian cell.

33. The method of one of claims 1 to 32, wherein the eukaryotic cell expresses the taste receptor, a subunit of the taste receptor, or the G protein from an endogenous gene.

34. The method of any one of claims 1 to 33, wherein the eukaryotic cell is a member of a cell line selected from the group consisting of: Panc-1, ASPC-1, BxPc-3, HuTu-80, HCT-8, HT-29, SW48, COLO205, COLO320-DM, HCT-15, NCI-H716, CCD841 CoN, CCD-112 CoN, and CCD-33Co.

35. The method of any one of claims 1 to 34, wherein the eukaryotic cell is a human cell or a non-human primate cell, and wherein the eukaryotic cell endogenously expresses a functional TAS1R or TAS2R receptor.

36. The method of claim 35, wherein the eukaryotic cell is a human cell, and wherein the human cell is an exocrine secretory epithelial cell, a hormone secreting cell, an epithelial cell, a ciliated cell, a keratinocyte, a sensory transducer cell, an autonomic neuron cell, a central nervous system neuron, a lens cell, a hepatocyte, an adipocyte, a lipocyte, a barrier function cell, an extracellular matrix cell, a contractile cell, a blood cell, an immune cell, a pigment cell, a germ cell, a nurse cell, an interstitial cell, or a mucus cell.

37. The method of any one of claims 1 to 36, wherein the eukaryotic cell is transformed with a construct that provides for the expression of the G protein, the TAS1R protein, the TAS2R protein, or any combination thereof.

38. The method of any one of claims 1 to 37, wherein the eukaryotic cell is a human cell, a mouse cell, a rat cell, or a non-human primate cell.

39. The method of any one of claims 1 to 38, wherein the TAS1R protein, the TAS2R protein, or any combination thereof are a human protein, a non-human primate protein, a mouse protein, a rat protein, a cat protein, or a dog protein.

40. The method of one of claims 1 to 40, wherein the taste receptor comprises a chimeric protein selected from the group consisting of a chimeric TAS1R protein, a chimeric TAS2R protein, a chimeric G protein, any combination thereof.

41. The method of claim 40, wherein the taste receptor comprises two or more chimeric proteins, and wherein the two or more chimeric proteins originate from at least two orthologs of a same gene, which is a TAS1R gene or a TAS2R gene.

42. The method of claim 40 or 41, wherein the chimeric protein comprises at least one extracellular, transmembrane, or intracellular domain of a gene, which is a TAS1R gene or a TAS2R gene.

43. The method of claim any one of claims 40 to 42, wherein the chimeric protein further comprises a domain of an additional G-coupled protein receptor, which is different from that of the G-coupled protein receptor, and wherein the domain is an extracellular domain, a transmembrane domain, or an intracellular domain.

44. The method of any one of claims 40 to 43, wherein the chimeric protein comprises a transmembrane domain of a TAS1R gene and an extracellular domain of a calcium sensing receptor gene.

45. The method of any one of claims 40 to 44, wherein the chimeric protein comprises an extracellular domain of a TAS1R gene and a transmembrane domain of a calcium sensing receptor gene.

46. The method of any one of claims 1 to 45, wherein the taste receptor comprises a polypeptide having at least 80% identity, or at least 85% identity, or at least 90% identity, or at least 93% identity, or at least 95% identity, or at least 97% identity, or at least 98% identity, or at least 99% identity, or 100% identity, to a wild-type TAS1R protein, a wild-type TAS2R protein, or a fragment or a domain thereof.

47. The method of any one of claims 1 to 46, wherein the eukaryotic cell is contained in a multiwell assay plate.

48. The method of any one of claims 1 to 47, wherein the eukaryotic cell is adhered to a substrate.

49. The method of any one of claims 1 to 48, wherein the method is a high throughput screening assay.

50. A compound, wherein the compound is the taste receptor modulator identified by the method of any one of claims 1 to 49.

51. Use of the taste receptor modulator identified by the method of any one of claims 1 to 49 to modify a taste of a comestible composition.

52. The use of claim 51, wherein the comestible composition is not a naturally occurring composition.

53. The use of claim 51, wherein the comestible composition is a naturally occurring composition.

54. The use of any one of claims 51 to 53, wherein modifying the taste of a comestible composition comprises enhancing a sweet taste of the comestible composition, enhancing an umami taste of a comestible composition, or inhibiting a bitter taste of a comestible composition.

55. The use of any one of claims 51 to 54, wherein the comestible composition is a food product, a beverage product, a pharmaceutical composition, a cosmetic composition, an oral care composition, or a dentrifice.

56. A comestible composition comprising the taste receptor modulator identified by the method of any one of claims 1 to 49.

57. The comestible composition of claim 56, which is not a naturally occurring composition.

58. The comestible composition of claim 55, which is a naturally occurring composition.

59. The comestible composition of claim 55 or 56, which is a food product, a beverage product, a pharmaceutical composition, a cosmetic composition, an oral care composition, or a dentrifice.


SEQUENCE LISTING (25 sequences)

SEQ ID NO: 1 - protein, 841 aa, Homo sapiens
MLLCTARLVGLQLLISCCWAFACHSTESSPDFTLPGDYLLAGLFPLHSGCLQVRHRPEVTLCDRSCSFNEHGYHLFQAMRLGVEEINNSTALLPNITLGYQLYDVCSDSANVYATLRVLSLPGQHHIELQGDLLHYSPTVLAVIGPDSTNRAATTAALLSPFLVPMISYAASSETLSVKRQYPSFLRTIPNDKYQVETMVLLLQKFGWTWISLVGSSDDYGQLGVQALENQATGQGICIAFKDIMPFSAQVGDERMQCLMRHLAQAGATVVVVFSSRQLARVFFESVVLTNLTGKVWVASEAWALSRHITGVPGIQRIGMVLGVAIQKRAVPGLKAFEEAYARADKKAPRPCHKGSWCSSNQLCRECQAFMAHTMPKLKAFSMSSAYNAYRAVYAVAHGLHQLLGCASGACSRGRVYPWQLLEQIHKVHFLLHKDTVAFNDNRDPLSSYNIIAWDWNGPKWTFTVLGSSTWSPVQLNINETKIQWHGKDNQVPKSVCSSDCLEGHQRVVTGFHHCCFECVPCGAGTFLNKSDLYRCQPCGKEEWAPEGSQTCFPRTVVFLALREHTSWVLLAANTLLLLLLLGTAGLFAWHLDTPVVRSAGGRLCFLMLGSLAAGSGSLYGFFGEPTRPACLLRQALFALGFTIFLSCLTVRSFQLIIIFKFSTKVPTFYHAWVQNHGAGLFVMISSAAQLLICLTWLVVWTPLPAREYQRFPHLVMLECTETNSLGFILAFLYNGLLSISAFACSYLGKDLPENYNEAKCVTFSLLFNFVSWIAFFTTASVYDGKYLPAANMMAGLSSLSSGFGGYFLPKCYVILCRPDLNSTEHFQASIQDYTRRCGST

SEQ ID NO: 2 - protein, 839 aa, Homo sapiens
MGPRAKTICSLFFLLWVLAEPAENSDFYLPGDYLLGGLFSLHANMKGIVHLNFLQVPMCKEYEVKVIGYNLMQAMRFAVEEINNDSSLLPGVLLGYEIVDVCYISNNVQPVLYFLAHEDNLLPIQEDYSNYISRVVAVIGPDNSESVMTVANFLSLFLLPQITYSAISDELRDKVRFPALLRTTPSADHHVEAMVQLMLHFRWNWIIVLVSSDTYGRDNGQLLGERVARRDICIAFQETLPTLQPNQNMTSEERQRLVTIVDKLQQSTARVVVVFSPDLTLYHFFNEVLRQNFTGAVWIASESWAIDPVLHNLTELGHLGTFLGITIQSVPIPGFSEFREWGPQAGPPPLSRTSQSYTCNQECDNCLNATLSFNTILRLSGERVVYSVYSAVYAVAHALHSLLGCDKSTCTKRVVYPWQLLEEIWKVNFTLLDHQIFFDPQGDVALHLEIVQWQWDRSQNPFQSVASYYPLQRQLKNIQDISWHTVNNTIPMSMCSKRCQSGQKKKPVGIHVCCFECIDCLPGTFLNHTEDEYECQACPNNEWSYQSETSCFKRQLVFLEWHEAPTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 3 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 4 - protein, 299 aa, Homo sapiens
MLESHLIIYFLLAVIQFLLGIFTNGIIVVVNGIDLIKHRKMAPLDLLLSCLAVSRIFLQLFIFYVNVIVIFFIEFIMCSANCAILLFINELELWLATWLGVFYCAKVASVRHPLFIWLKMRISKLVPWMILGSLLYVSMICVFHSKYAGFMVPYFLRKFFSQNATIQKEDTLAIQIFSFVAEFSVPLLIFLFAVLLLIFSLGRHTRQMRNTVAGSRVPGRGAPISALLSILSFLILYFSHCMIKVFLSSLKFHIRRFIFLFFILVIGIYPSGHSLILILGNPKLKQNAKKFLLHSKCCQ

SEQ ID NO: 5 - protein, 316 aa, Homo sapiens
MMGLTEGVFLILSGTQFTLGILVNCFIELVNGSSWFKTKRMSLSDFIITTLALLRIILLCIILTDSFLIEFSPNTHDSGIIMQIIDVSWTFTNHLSIWLATCLGVLYCLKIASFSHPTFLWLKWRVSRVMVWMLLGALLLSCGSTASLINEFKLYSVFRGIEATRNVTEHFRKKRSEYYLIHVLGTLWYLPPLIVSLASYSLLIFSLGRHTRQMLQNGTSSRDPTTEAHKRAIRIILSFFFLFLLYFLAFLIASFGNFLPKTKMAKMIGEVMTMFYPAGHSFILILGNSKLKQTFVVMLRCESGHLKPGSKGPIFS

SEQ ID NO: 6 - protein, 299 aa, Homo sapiens
MLRLFYFSAIIASVILNFVGIIMNLFITVVNCKTWVKSHRISSSDRILFSLGITRFLMLGLFLVNTIYFVSSNTERSVYLSAFFVLCFMFLDSSSVWFVTLLNILYCVKITNFQHSVFLLLKRNISPKIPRLLLACVLISAFTTCLYITLSQASPFPELVTTRNNTSFNISEGILSLVVSLVLSSSLQFIINVTSASLLIHSLRRHIQKMQKNATGFWNPQTEAHVGAMKLMVYFLILYIPYSVATLVQYLPFYAGMDMGTKSICLIFATLYSPGHSVLIIITHPKLKTTAKKILCFKK

SEQ ID NO: 7 - protein, 299 aa, Homo sapiens [sequence omitted]

SEQ ID NO: 8 - protein, 318 aa, Homo sapiens
MADKVQTTLLFLAVGEFSVGILGNAFIGLVNCMDWVKKRKIASIDLILTSLAISRICLLCVILLDCFILVLYPDVYATGKEMRIIDFFWTLTNHLSIWFATCLSIYYFFKIGNFFHPLFLWMKWRIDRVISWILLGCVVLSVFISLPATENLNADFRFCVKAKRKTNLTWSCRVNKTQHASTKLFLNLATLLPFCVCLMSFFLLILSLRRHIRRMQLSATGCRDPSTEAHVRALKAVISFLLLFIAYYLSFLIATSSYFMPETELAVIFGESIALIYPSSHSFILILGNNKLRHASLKVIWKVMSILKGRKFQQHKQI

SEQ ID NO: 9 - protein, 308 aa, Homo sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACM

SEQ ID NO: 10 - protein, 312 aa, Homo sapiens [sequence omitted]

SEQ ID NO: 11 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKMKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 12 - protein, 303 aa, Homo sapiens [sequence omitted]

SEQ ID NO: 13 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 14 - protein, 291 aa, Homo sapiens
MIPIQLTVFFMIIYVLESLTIIVQSSLIVAVLGREWLQVRRLMPVDMILISLGISRFCLQWASMLNNFCSYFNLNYVLCNLTITWEFFNILTFWLNSLLTVFYCIKVSSFTHHIFLWLRWRILRLFPWILLGSLMITCVTIIPSAIGNYIQIQLLTMEHLPRNSTVTDKLENFHQYQFQAHTVALVIPFILFLASTIFLMASLTKQIQHHSTGHCNPSMKAHFTALRSLAVLFIVFTSYFLTILITIIGTLFDKRCWLWVWEAFVYAFILMHSTSLMLSSPTLKRILKGKC

SEQ ID NO: 15 - protein, 319 aa, Homo sapiens
MITFLPIIFSILIVVIFVIGNFANGFIALVNSIEWVKRQKISFVDQILTALAVSRVGLLWVLLLHWYATQLNPAFYSVEVRITAYNVWAVTNHFSSWLATSLSMFYLLRIANFSNLIFLRIKRRVKSVVLVILLGPLLFLVCHLFVINMDETVWTKEYEGNVTWKIKLRSAMYHSNMTLTMLANFVPLTLTLISFLLLICSLCKHLKKMQLHGKGSQDPSTKVHIKALQTVTSFLLLCAIYFLSMIISVCNLGRLEKQPVFMFCQAIIFSYPSTHPFILILGNKKLKQIFLSVLRHVRYWVKDRSLRLHRFTRGALCVF

SEQ ID NO: 16 - protein, 333 aa, Homo sapiens [sequence omitted]

SEQ ID NO: 17 - protein, 321 aa, Homo sapiens
MTKLCDPAESELSPFLITLILAVLLAEYLIGIIANGFIMAIHAAEWVQNKAVSTSGRILVFLSVSRIALQSLMMLEITISSTSLSFYSEDAVYYAFKISFIFLNFCSLWFAAWLSFFYFVKIANFSYPLFLKLRWRITGLIPWLLWLSVFISFSHSMFCINICTVYCNNSFPIHSSNSTKKTYLSEINVVGLAFFFNLGIVTPLIMFILTATLLILSLKRHTLHMGSNATGSNDPSMEAHMGAIKAISYFLILYIFNAVALFIYLSNMFDINSLWNNLCQIIMAAYPASHSILLIQDNPGLRRAWKRLQLRLHLYPKEWTL

SEQ ID NO: 18 - protein, 323 aa, Homo sapiens [sequence omitted]

SEQ ID NO: 19 - protein, 309 aa, Homo sapiens
MITFLPIIFSSLVVVTFVIGNFANGFIALVNSIEWFKRQKISFADQILTALAVSRVGLLWVLLLNWYSTVLNPAFNSVEVRTTAYNIWAVINHFSNWLATTLSIFYLLKIANFSNFIFLHLKRRVKSVILVMLLGPLLFLACHLFVINMNEIVRTKEFEGNMTWKIKLKSAMYFSNMTVTMVANLVPFTLTLLSFMLLICSLCKHLKKMQLHGKGSQDPSTKVHIKALQTVISFLLLCAIYFLSIMISVWSFGSLENKPVFMFCKAIRFSYPSIHPFILIWGNKKLKQTFLSVFWQMRYWVKGEKTSSP

SEQ ID NO: 20 - protein, 309 aa, Homo sapiens
MMSFLHIVFSILVVVAFILGNFANGFIALINFIAWVKRQKISSADQIIAALAVSRVGLLWVILLHWYSTVLNPTSSNLKVIIFISNAWAVTNHFSIWLATSLSIFYLLKIVNFSRLIFHHLKRKAKSVVLVIVLGSLFFLVCHLVMKHTYINVWTEECEGNVTWKIKLRNAMHLSNLTVAMLANLIPFTLTLISFLLLIYSLCKHLKKMQLHGKGSQDPSTKIHIKALQTVTSFLILLAIYFLCLIISFWNFKMRPKEIVLMLCQAFGIIYPSFHSFILIWGNKTLKQTFLSVLWQVTCWAKGQNQSTP

SEQ ID NO: 21 - protein, 309 aa, Homo sapiens
MTTFIPIIFSSVVVVLFVIGNFANGFIALVNSIEWVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNMTWKIKLRSAVYLSDATVTTLGNLVPFTLTLLCFLLLICSLCKHLKKMQLHGKGSQDPSTKVHIKVLQTVIFFLLLCAIYFLSIMISVWSFGSLENKPVFMFCKAIRFSYPSIHPFILIWGNKKLKQTFLSVLRQVRYWVKGEKPSSP

SEQ ID NO: 22 - protein, 299 aa, Homo sapiens
MMCFLLIISSILVVFAFVLGNVANGFIALVNIIDWVNTRKISSAEQILTALVVSRIGLLWVMLFLWYATVFNSALYGLEVRIVASNAWAVTNHFSMWLAASLSIFCLLKIANFSNLISLHLKKRIKSVVLVILLGPLVFLICNLAVITMDERVWTKEYEGNVTWKIKLRNAIHLSSLTVTTLANLIPFTLSLICFLLLICSLCKHLKKMRLHSKGSQDPSTKVHIKALQTVTSFLMLFAIYFLCIITSTWNLRTQQSKLVLLLCQTVAIMYPSFHSFILIMGSRKLKQTFLSVLWQMTR

SEQ ID NO: 23 - protein, 299 aa, Homo sapiens
MITFLYIFFSILIMVLFVLGNFANGFIALVNFIDWVKRKKISSADQILTALAVSRIGLLWALLLNWYLTVLNPAFYSVELRITSYNAWVVTNHFSMWLAANLSIFYLLKIANFSNLLFLHLKRRVRSVILVILLGTLIFLVCHLLVANMDESMWAEEYEGNMTGKMKLRNTVHLSYLTVTTLWSFIPFTLSLISFLMLICSLCKHLKKMQLHGEGSQDLSTKVHIKALQTLISFLLLCAIFFLFLIVSVWSPRRLRNDPVVMVSKAVGNIYLAFDSFILIWRTKKLKHTFLLILCQIRC

SEQ ID NO: 24 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFPGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSTQAHTRALKSLISFLILYALSFLSLIIDAAKFISMQNDFYWPWQIAVYLCISVHPFILIFSNLKLRSVFSQLLLLARGFWVA

SEQ ID NO: 25 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS